(19) United States
(12) Patent Application Publication
(10) Pub. No.: US 2019/0038498 A1
(43) Pub. Date: Feb. 7, 2019

(54) METHODS OF TREATING AND PREVENTING DISEASE BY CLAMPING OF SKIN

(71) Applicant: Natural Pharmacia International, Inc., Burlington, MA (US)

(72) Inventors: David Yue-Wei LEE, Cambridge, MA (US); Long Zhang CHEN, Beijing (CN)

(21) Appl. No.: 16/081,128

(22) PCT Filed: Feb. 28, 2017
(86) PCT No.: PCT/US17/19855

§ 371 Date: Aug. 30, 2018

Related U.S. Application Data
(60) Provisional application No. 62/301,850, filed on Mar. 1, 2016.

(51) Int. Cl. A61H 7/00; A61B 17/28

(52) U.S. Cl. CPC A61H 7/00; B25B 7/18; A61B 17/28

(57) ABSTRACT

The invention provides methods of treating diseases, such as various cancers, neoplasms, autoimmune diseases, and infectious diseases, among others, by clamping the skin of a subject in need of treatment so as to apply a compressive force that is sufficient to cause damage, often manifested in pain or bruising, to the underlying tissue. The invention additionally provides kits containing clamps capable of regulating the distance between the arms of the clamp such that a user of the clamp will apply a safe amount of pressure to the skin of a subject that is sufficient to cause tissue bruising and/or pain.

### FIELD OF THE INVENTION

The invention relates to methods of treating diseases, such as various cancers, autoimmune diseases, and infectious diseases, as well as to promote health in a subject by clamping of skin.

### BACKGROUND OF THE INVENTION

Cancers, autoimmune diseases, and infectious diseases represent pathologies that have been notoriously difficult to target using conventional therapeutics, as current treatment modalities for these conditions typically employ combinations of small molecules or complex biologic agents to modulate disease progression. Conventional therapeutics have targeted these diseases with mixed results. Small molecule therapeutics often elicit toxic responses due to off-target effects and harmful intermediates produced during drug metabolism, while biologic agents have frequently encountered difficulty gaining traction in clinical applications due to poor bioavailability and stability in human serum. The immune system provides an array of defense mechanisms that are used in nature to prevent, mitigate, and eradicate various disease-causing agents, but has yet to be harnessed successfully to treat human disease without the administration of exogenous pharmacological materials. There currently exists a need for a methodology capable of stimulating innate and adaptive immune activity to treat various human pathologies and to maintain healthy conditions.

Currently, a variety of therapeutic techniques for treating cancer are being developed. These include cancer immunotherapies, such as chimeric antigen receptor T-cell (CAR T) therapy, that function by reprogramming a patient's own immune cells so as to mount an immune response against cancerous cells. Other cancer treatment techniques include the administration of anti-CTLA-4 therapeutics, such as ipilimumab, which function by attenuating the inactivation of T-cells, thus allowing these cells to execute a full immune attack. Additional cancer treatment techniques include anti-PD-1 therapy, as well as dendritic cell vaccines. The financial expense of these immunotherapies can be prohibitive, as these treatment options can cost nearly half million dollars per patient due to the complexity of the biochemical procedures involved. Moreover, many of these therapeutic modalities are still in the development stage and have not yet been commercially implemented.

There exists a need for a therapeutic technique that can be used to target a broad spectrum of disease, including cancers and infectious diseases, in a manner that is inexpensive and readily accessible.

### SUMMARY OF THE INVENTION

The present invention provides methods for treating various human disorders, such as cancers, autoimmune diseases, infectious diseases, elevated serum glucose and lipid conditions, neuromuscular diseases, as well as renal, lung, and liver diseases, among others, by clamping the skin and underlying tissue of a subject in need of treatment so as to apply a compressive force sufficient to cause damage to the tissue. This damage may manifest as bruises and/or pain at the site of clamping, which can be an indication that the inflammatory immune response has been induced. The effects of clamping therapy additionally include the activation of antigen-presenting immune cells, such as dendritic cells, as well as T- and B-lymphocytes capable of mounting cell-mediated and humoral immune responses against antigens associated with the disease being treated. The invention additionally provides kits containing clamps that can be used to treat a subject in need of compressile therapy. Preferred clamps include those that have internal regulators capable of modulating the distance between the arms of the clamp, such that the arms can be adjusted so as to remain close to selected distances that allow the application of compressile force sufficient to cause tissue bruising and pain without causing irreparable bodily harm.

In one aspect, the invention provides a method of treating a disease in a subject by enclosing a portion of tissue that includes the skin of the subject within a clamp and clamping the tissue for a duration and with sufficient pressure to cause damage to the tissue. For instance, in some embodiments the method includes the steps of compressing a portion of tissue containing skin of the subject by enclosing the skin within a clamp and applying pressure to the skin by clamping sufficient to cause damage to the tissue.

In some embodiments, the method includes folding the portion to form a crease, e.g., prior to the enclosing described above, for instance, by lifting the skin to form a region of skin that is convex with respect to surrounding skin that is not folded.

In some embodiments, the portion of tissue includes one or more tumors. In these instances, the clamping may cause crushing of the one or more tumors.

In some embodiments, the method includes compressing one or more muscles and/or connective tissues located underneath the skin. In some embodiments, the pressure is applied at a location at which the skin converges with surrounding skin that is not folded.

In another embodiment, the clamp contains curved ends and a flexible handle. For instance, the flexible handle may be configured to allow a user of the clamp to control the duration of pressure applied by the clamping. In some embodiments, the flexible handle is configured to allow a user of the clamp to control the amount of pressure applied by the clamping.

In another embodiment, the clamp contains a mechanism capable of regulating the distance between the curved ends. For instance, the clamp may contain a regulator screw capable of maintaining a desired distance between arms of the clamp. In some embodiments, the regulator screw can be adjusted to one of a plurality of settings (e.g., one of three settings). These settings may correspond to a state of maximum distance between the arms of the clamp (e.g., from about 2.4 cm to about 7.2 cm), one or more states of intermediate distance between the arms of the clamp (e.g., from about 1.6 cm to about 4.8 cm), and a state of minimum distance between the arms of the clamp (e.g., from about 0.8 cm to about 2.4 cm). In some embodiments, the setting that corresponds to the state of maximum distance between the arms of the clamp is indicated by exposure of a green color adjacent to an arm of the clamp. Additionally or alternatively, the setting that corresponds to the state of intermediate distance between the arms of the clamp may be indicated by exposure of a yellow color adjacent to an arm of the clamp. In some embodiments, the setting that corresponds to the state of minimum distance between the arms of the clamp is indicated by exposure of a red color adjacent to an arm of the clamp.

In some embodiments of the invention, the clamping occurs for a period of about 1 to about 10 seconds (e.g., for a period of about 1 to 3 seconds). The clamping may be performed one or more times daily, weekly, monthly, or yearly. For instance, the clamping may be performed one or more times daily for between about two and about twenty days (e.g., the clamping may be performed one or more times daily for six days). In some embodiments, at the end of the six days, the clamping is repeated one or more times every other day for a total of 16 additional days to form a standard treatment course.

In another embodiment, the standard treatment course is repeated between two and twenty times (e.g., between five and fifteen times, such as between six and nine times). For instance, the standard treatment course may be repeated seven times. In other embodiments, the standard treatment course is repeated eight times. In some embodiments, the clamping is performed with increasing pressure over the duration of the standard treatment course.

In some embodiments, the clamping is sufficient to cause pain and/or bruising to the tissue. Additionally or alternatively, the clamping may cause an increase in the amount or concentration of one or more antigens on the surface of a cancer cell within the subject. In some embodiments, the clamping causes activation of, and/or an increase in the amount or concentration of, one or more dendritic cells at or near cancer tissue (e.g., bound to the surface of a cancer cell) within the subject. The dendritic cells may be located below the skin and may be activated (e.g., Langerhans cells).

In some embodiments, the subject exhibits an elevated immune response after the clamping is performed. For instance, the enhanced immune response may include inflammation, secretion of chemokines, an increase in the level of one or more cytokines, and/or activation of one or more immune cells within the subject. In some embodiments, the inflammation includes one or more processes selected from the group consisting of arteriole dilation, an increase in capillary permeability, and migration of neutrophils and/or macrophages from capillaries or venules into interstitial spaces. Additionally or alternatively, the chemokines may be capable of attracting to the portion of skin one or more macrophages, T-cells (e.g., CD4+ or CD8+ T-cells), mast cells, dendritic cells, activated dendritic cells, eosinophils, and/or neutrophils. In some embodiments, the one or more cytokines are selected from the group consisting of TNFα, IFNγ, IL-1, IL-6, and IL-8. In some embodiments, the one or more immune cells are selected from the group consisting of macrophages, neutrophils, T-cells (e.g., CD4+ or CD8+ T-cells), antigen-presenting cells, and dendritic cells.

The clamping of any of the foregoing embodiments of the invention may cause the subject to exhibit epidermal lumps after the clamping is performed. For instance, the lumps may be between about 1 cm and about 3 cm in diameter. The method may further include the step of compressing the lumps, e.g., such that the lumps are broken into a plurality of fragments. In some embodiments, the lumps contain cell debris, such as monosodium urate. The monosodium urate may be in a crystalline form. In some embodiments, the compressing causes the monosodium urate to be released within the subject (e.g., into peripheral tissues within the subject).

In some embodiments, the monosodium urate stimulates the growth and/or maturation of one or more cells, such as macrophages, neutrophils, T-cells (e.g., CD4+ or CD8+ T-cells), antigen-presenting cells, and/or dendritic cells. The monosodium urate may induce the production of IL-1β in the subject. In some embodiments, the monosodium urate promotes the growth or maturation of one or more cells selected from the group consisting of macrophages, neutrophils, T-cells, antigen-presenting cells, and dendritic cells. In some embodiments of the invention, the T-cells are capable of specifically binding an antigen expressed on the surface of a cancer cell and/or killing one or more of the cancer cells.

In some embodiments, the skin that is clamped is located along a meridian line of the subject. The meridian line may be selected from the group consisting of wood phase meridian, first fire phase meridian, second fire phase meridian, earth phase meridian, metal phase meridian, water phase meridian, lung meridian, heart meridian, liver meridian, spleen meridian, kidney meridian, pericardium meridian, large intestine meridian, small intestine meridian, stomach meridian, bladder meridian, and gall bladder meridian.

In some embodiments, the site of disease is identified and clamping is subsequently performed along meridian lines nearest the site of disease. For instance, the disease may be a cancer, in which case the clamping may be performed along meridian lines nearest one or more solid tumors in the patient. In some embodiments, the disease is an infectious disease, and the clamping is subsequently performed along meridian lines nearest the site of the infection (e.g., nearest the site of a manifestation of the infection, such as a rash, swelling, inflammation, itch, pain, redness, edema, etc.).

In some embodiments of the invention, the disease that is treated is a cancer, such as a cancer selected from the group consisting of leukemia, lymphoma, liver cancer, bone cancer, skin cancer, pulmonary cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, adenocarcinoma, pituitary adenoma, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrioma, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), ovarian carcinoma, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, uterine sarcoma, vaginal cancer, vulvar cancer, and waldenström macroglobulinemia. In some embodiments, the cancer is selected from the group consisting of breast cancer (e.g., late stage breast cancer, bilateral breast ductal carcinoma, or invasive bilateral breast cancer), adenocarcinoma, leukemia, skin cancer, ovarian carcinoma, pituitary adenoma, pulmonary cancer, endometrioma, and cervical cancer.

In some embodiments, the disease that is treated is a neoplasm. The neoplasm may be a growth of a tissue or organ selected the group consisting of a pancreas, salivary gland, pituitary gland, kidney, heart, lung, hematopoietic system, cranial nerves, heart, aorta, olfactory gland, hypopharynx, ear, nerves, structures of the head, eye, thymus, tongue, bone, liver, small intestine, large intestine, gut, brain, skin, peripheral nervous system, central nervous system, spinal cord, breast, embryonic structures, embryos, and testes. In some embodiments, the neoplasm is a growth of an organ selected from the group consisting of a lung, kidney, and hypopharynx.

In some embodiments, the disease is an autoimmune disease selected from the group consisting of type I diabetes, alopecia areata, ankylosing spondylitis, antiphospholipid syndrome, autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, crest syndrome, cold agglutinin disease, Crohn's Disease, essential mixed cryoglobulinemia, fibromyalgia-fibromyositis, Graves' Disease, Guillain-Barré Syndrome, Hashimoto's thyroiditis, hypothyroidism, idiopathic pulmonary fibrosis, idiopathic thrombocytopenia purpura (ITP), IgA nephropathy, juvenile arthritis, lichen planus, lupus, systemic lupus erythematosus, Ménière's Disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, stiff-man syndrome, Takayasu Arteritis, temporal arteritis/giant cell arteritis, ulcerative colitis, uveitis, vasculitis, vitiligo, and Wegener's Granulomatosis.

In some embodiments, the autoimmune disease is diabetes (e.g., type I diabetes) or lupus.

In some embodiments, the disease is an infectious disease caused by one or more agents selected from the group consisting of a virus, a bacterium, a fungus, or a parasite. For instance, the virus may be selected from the group consisting of Gadgets Gully virus, Kadam virus, Kyasanur Forest disease virus, Langat virus, Omsk hemorrhagic fever virus, Powassan virus, Royal Farm virus, tick-borne encephalitis virus, Louping ill virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, and Rio Bravo virus, Venezuelan equine encephalitis virus (VEE), Eastern equine encephalitis virus (EEE), Western equine encephalitis virus (WEE), Ebola virus, Marburg virus, smallpox virus, vaccinia virus, Lassa virus, Ippy virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, and Whitewater Arroyo virus, Sin Nombre virus, Hantaan virus, Rift Valley fever virus, Crimean-Congo hemorrhagic fever virus, Dugbe virus, herpes simplex virus (HSV), cytomegalovirus (CMV), Epstein-Barr virus (EBV), Kaposi's sarcoma associated-herpesvirus (KSHV), influenzavirus A, H5N1 avian influenza virus, influenzavirus B, influenzavirus C, severe acute respiratory syndrome (SARS) virus, rabies virus, and vesicular stomatitis virus (VSV).

In some embodiments, the bacterium is selected from the group consisting of Pseudomonas aeruginosa, Salmonella typhimurium, Escherichia coli, Klebsiella pneumoniae, Bruscella, Burkholderia mallei, Yersinia pestis, and Bacillus anthracis.

In some embodiments, the fungus is selected from the group consisting of Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, and Rhizopus arrhizus.

In some embodiments, the parasite is selected from the group consisting of Toxoplasma Plasmodium falciparum, P. vivax, P. ovale, P. malariae, Trypanosoma spp., and Legionella spp.

In some embodiments, the disease is selected from the group consisting of hypertension, hyperglycemia, hyperlipidemia, edema, obesity, infertility, amenorrhea, fatigue, vertigo, uterine bleeding, uterine ulcer, hyperthyroidism, myoma, endometriosis, cerebral palsy, brain atrophy, systemic muscular atrophy, trigeminal neuralgia, depression, schizophrenia, epilepsy, amyotrophic lateral sclerosis (ALS), Parkinson's Disease, autism, Alzheimer's Disease, Huntington's Disease, emphysema, asthma, hepatitis B, cough, systemic fibroma, renal diseases, lung diseases, and liver diseases.

In some embodiments of the above-described methods of the invention, the subject is a mammal, such as a human.

The methods of the invention can be used to treat the disease and/or to reduce or eliminate one or more symptoms thereof. For instance, in some embodiments, clamping therapy is administered to a patient that has been diagnosed as having an infectious disease, such as an infectious disease described herein, to reduce and/or eliminate one or more symptoms of the infectious disease (e.g., rash, swelling, inflammation, itch, pain, redness, edema, etc.).

In another aspect, the invention provides a kit containing a clamp and a package insert. The package insert may instruct a user of the kit to treat a subject according to any of the foregoing methods of the invention.

In some embodiments, the clamp contains a regulator screw capable of maintaining a desired distance between arms of the clamp. For instance, the regulator screw may be adjusted to one of a plurality of settings (e.g., one of three settings). In some embodiments, these settings correspond to a state of maximum distance between the arms of the clamp, one or more states of intermediate distance between the arms of the clamp, and a state of minimum distance between the arms of the clamp. In some embodiments, the setting that corresponds to the state of maximum distance between the arms of the clamp is indicated by exposure of a first color adjacent to an arm of the clamp. Additionally or alternatively, the setting that corresponds to the state of intermediate distance between the arms of the clamp may be indicated by exposure of a second color adjacent to an arm of the clamp. In some embodiments, the setting that corresponds to the state of minimum distance between the arms of the clamp is indicated by exposure of a third color adjacent to an arm of the clamp. In some embodiments, the first, second, and third colors are each different from one another. In some embodiments, the first color is green. In some embodiments, the second color is yellow. In some embodiments, the third color is red.

### Definitions

As used herein, the term “about” refers to a value that is within 10% above or below the value being described. For instance, “about 50 mg/dL” denotes a concentration that is from 45 mg/dL to 55 mg/dL.

As used herein, the term “clamping” refers to a procedure for applying pressure to a substrate by placing the substrate between two or more ends of a device and subsequently moving one or more arms of the device such that the two ends are brought into proximity with one another. This movement may position the two ends within a distance of one another that is sufficient for both ends to be in contact with the substrate, such as the skin and underlying tissue of a patient in need of clamping therapy as described herein. The movement of the two arms may result in a pressure of, e.g., 5 psi or more (e.g., 5 psi, 6 psi, 7 psi, 8 psi, 9 psi, 10 psi, 11 psi, 12 psi, 13 psi, 14 psi, 15 psi, 16 psi, 17 psi, 18 psi,19 psi, 20 psi, 25 psi, 30 psi, 35 psi, 40 psi, 45 psi, 50 psi, 55 psi, 60 psi, 65 psi, 70 psi, 75 psi, 80 psi, 85 psi, 90 psi, 95 psi, 100 psi, 110 psi, 120 psi, 130 psi,140 psi, 150 psi, 160 psi, 170 psi, 180 psi, 190 psi, 200 psi, 300 psi, 400 psi, 500 psi, 600 psi,700 psi, 800 psi, 900 psi, 1,000 psi, or more). In some embodiments, a clamp of the form shown in FIGS. 1A and 1B can be used to apply pressure to the skin and underlying tissue of a patient in need of therapy, e.g., in the manner portrayed in FIG. 10. A physician of skill in the art may fold the skin of the patient so as to lift the skin and underlying tissue above the surrounding skin and subsequently clamp the lifted skin. In preferred embodiments, the clamping is performed at the point at which the folded skin converges with the neighboring skin that is not folded by the physician, e.g., in a manner portrayed in FIG. 2 or FIG. 3. In some embodiments, a regulator screw (1) as shown in FIGS. 1B and 1C can be used to adjust/select the distance between the arms of the clamp in order to modulate the pressure that is applied to the patient as described herein.

As used herein, the term “cytokine” refers to a substance of a general class of biological molecules that modulate the activities of cells of the immune system. Cytokines include, but are not limited to, those biological molecules (e.g., proteins) that bind to and act on cells of a tissue that is proximal to the site of secretion, as well as those molecules that circulate in the blood so as to access tissues distal from those at which the cytokines originate. Exemplary cytokines that may be modulated in response to clamping therapy as described herein include, without limitation, IFNα, IFNβ, IFNγ, interleukins (e.g., IL-1 to IL-29, such as IL-1, IL-2, IL-5, IL-6, IL-7, IL-8, IL-10, IL-12, IL-15, and IL-18), tumor necrosis factors (e.g., TNFα and TNFβ), erythropoietin (EPO), monocyte chemotactic protein (MCP)-1, intracellular adhesion molecules (ICAMs), macrophage colony stimulating factor (M-CSF), granulocyte colony stimulating factor (G-CSF), and granulocyte-macrophage colony stimulating factor (GM-CSF).

As used herein, the terms “damage” and “tissue damage” refer to a physiological response in which one or more cells are killed or experience a state of stress, such as stress induced by a chemical stimulus (e.g., oxidative stress) or a physical action (e.g., compression). Damage also encompasses a state in which tissue is disrupted (e.g., skin displacement that may result in damage to blood vessels) or tissue architecture is disrupted (e.g., changes in skin turgor, edema, lesions, and erythema). In some embodiments, tissue damage may encompass apoptosis or necrosis of cells therein. Cells of a damaged tissue may also release chemical products, such as monosodium urate, which may crystallize, e.g., in the peripheral tissue of a patient undergoing clamping therapy. Tissue damage may also manifest in an inflammatory immune response, such as a release of inflammatory cytokines (e.g., tumor necrosis factor a (TNFα), interferon γ (IFNγ), interleukin-1 (IL-1), IL-6, and/or IL-8. In some embodiments, tissue damage can induce the activation of antigen-presenting cells, such as dendritic cells, cytotoxic T-cells, and/or Treg cells. In cancer patients undergoing clamping therapy, tissue damage may result in the activation of one or more dendritic cells and/or T-cells at the surface of a cancer cell or near cancerous tissue (e.g., at the surface of a solid tumor) to maximize immune functions.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted therefrom.

As used herein, the term “immune cell” refers to a cell that modulates a response to an exogenous substance or damage inflicted to a particular tissue. An immune cell, e.g., a Treg cell, may potentiate the activity of neighboring or peripheral cells, e.g., by synthesizing and secreting a cytokine, and/or by producing biomolecules (e.g., T-cell receptors, B-cell receptors, or immunoglobulins) that specifically bind a particular antigen, such as an antigen expressed on the surface of a cancer cell. Immune cells also include antigen-presenting cells capable of processing a target antigen and presenting the antigen, or a fragment thereof, on the surface of the cell, e.g., bound to a major histocompatability complex (MHC) class I or class II protein. Immune cells include those of hematopoietic origin, such as lymphocytes (e.g., T-cells, such as CD4+ Th1 cell or Th2 cells, CD8+ T-cells, such as cytotoxic T-cells, Treg cells, B-cells, and natural killer (NK) cells), as well as myeloid cells, such as monocytes, macrophages, dendritic cells, eosinophils, mast cells, basophils, and granulocytes, among others.

As used herein, the terms “meridian line” and “meridian” are used interchangeably and refer to a line segment on the human body that demarcates the meridian network. The meridian network is a collection of lines that coincide with various major blood vessels and organs. The meridian network is divided into two sets of meridian lines: the jingmai or meridian channels and the luomai or associated vessels (also referred to as “collaterals”). The jingmai contain the 12 tendinomuscular meridians, the 12 divergent meridians, the 12 principal meridians, the 8 extraordinary vessels, and the Huato channel, which is a set of bilateral points on the lower back. The collaterals contain 15 major arteries that intersect with and connect the 12 principal meridians, as well as their associated internal organs and other related internal structures. The collateral system also contains a network of capillaries which spread throughout the body, e.g., in the 12 cutaneous regions as well as emanating from each point on the principal meridians. Exemplary meridian lines are illustrated in FIG. 6.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject. Preferably, the sample is blood, a blood component (e.g., serum or plasma), or urine.

As used herein, the phrase “specifically binds” refers to a binding reaction which is determinative of the presence of a particular molecule (e.g., a cell-surface protein, antibody, receptor, or other biological molecule) within a heterogeneous population of compounds that is recognized by another molecule (e.g., a ligand, antibody, receptor, or other biological molecule) with particularity. A first molecule that specifically binds to a second molecule will bind to the second molecule with a K_{D} of less than 100 nM. For example, a ligand that specifically binds to a receptor will bind to the receptor with a K_{D} of up to 100 nM (e.g., between 1 pM and 100 nM). A ligand that does not exhibit specific binding to a receptor or a domain thereof will exhibit a K_{D} of greater than 100 nM (e.g., greater than 500 nm, 1 μM, 100 μM, 500 μM, or 1 mM) for that particular receptor or domain thereof. A variety of assay formats may be used to select ligands, antibodies, cells containing surface-exposed proteins, and other biological molecules that specifically bind to a particular target molecule. For example, solid-phase ELISA assays are routinely used to select ligands that specifically bind a receptor. See, Harlow & Lane, Antibodies, A Laboratory

Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” are interchangeable and refer to an organism that receives treatment for a particular disease or condition as described herein (such as a cancer, autoimmune disease, or infectious disease) or that is diagnosed as having a disease or condition. Examples of subjects and patients include mammals, such as humans, primates, pigs, goats, rabbits, hamsters, cats, dogs, guinea pigs, members of the bovidae family (such as cattle, bison, buffalo, and yaks, among others), cows, sheep, horses, and bison, among others, receiving treatment for diseases or conditions, for example, cancer, autoimmune diseases, infectious diseases, and diseases associated with a particular organ, such as kidney disease or liver disease.

As used herein, the terms “treat” or “treatment” refer to therapeutic treatment, in which the object is to prevent, slow down (lessen), or eliminate (e.g., remove one or more symptoms of, or all sequelae of) an undesired physiological change or disorder, such as cancer, an infectious disease, or an autoimmune disease. Beneficial or desired clinical results include, but are not limited to, alleviation of symptoms, diminishment of extent of disease, stabilized (i.e. , not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in which the condition or disorder is to be prevented.

### BRIEF DESCRIPTION OF THE FIGURES

FIG. 1A is an image showing a representative clamp that may be used in conjunction with the methods and kits of the invention. The clamp contains two arms that, when moved into close proximity with one another, induce the movement of the two ends of the clamp (shown outlined in dashed lines at the top of FIG. 1A) towards one another. The movement of the two ends into proximity with one another can cause pressure to be applied to a substance (e.g., the skin and underlying tissue of a patient receiving therapy) that is placed between the two ends.

FIG. 1B is a diagram showing a clamp that contains a mechanism for regulating the distance between the two arms of the clamp. The screw shown in FIG. 1B can be adjusted such that the portion of the screw that is situated between the two arms of the clamp is longer (e.g., such that the distance between the arms of the clamp is maximized), shorter (e.g., such that the distance between the arms of the clamp is minimized), or intermediate (e.g., such that the distance between the arms of the clamp is between the maximum and minimum distances) so as to modulate the minimum distance between the arms of the clamp (i.e., the distance between the arms when the clamp is in a fully-closed state). This adjustment serves to regulate the pressure that can be applied by the clamp, as longer minimum distances between the arms of the clamp result in longer minimum distances between the ends of the clamp when brought in contact with a substrate, such as the skin and underlying tissue of a patient receiving clamping therapy. The longer minimum distances between the ends of the clamp deliver less pressure to a patient than intermediate or shorter minimum distances between the ends of the clamp. In this way, a physician of skill in the art can regulate the amount of pressure delivered to a patient. For instance, a physician may deliver less pressure during initial stages of treatment by clamping the skin and underlying tissue of a patient using a clamp that is maintained in a state of maximum distance between the arms, e.g., by adjusting the regulator screw such that the distance between the two arms of the clamp is maximized. A physician may gradually decrease the distance between the arms of the clamp as the therapy regimen continues, e.g., over the course of days, weeks, months, or years, by adjusting the regulator screw such that there is an intermediate distance between the two arms of the clamp. As treatment progresses, a physician may adjust the regulator screw such that the distance between the arms of the clamp is minimized. This allows a user of the clamp to bring the arms of the clamp into closer proximity, thus enabling the ends of the clamp to apply greater pressure to, e.g., the skin and underlying tissue of a patient undergoing clamping therapy. Using this treatment regime, a physician may ensure that safe amounts of pressure are applied to the skin and underlying tissue of a patient while maintaining therapeutic efficacy.

FIG. 1C is a diagram showing clamping therapy being performed on a patient when the distance between the two arms of the clamp is maintained at an intermediate level by virtue of the regulator screw having been adjusted to a state in which the yellow region, but not the green region, of the screw is between the two arms of the clamp.

FIG. 2 is a photograph showing a physician administering clamping therapy to a patient at the patient's right arm. The physician has folded the skin of the patient so as to lift the skin and underlying tissue above the neighboring skin as to form a convex protrusion. Pressure is then applied to the protruded skin and tissue beneath the epidermal surface by clamping the skin as shown. The clamping may take place for a duration of, e.g., from 1 to 3 seconds.

FIG. 3 is a photograph showing a physician administering clamping therapy to a patient at the patient's lower back. The physician has folded the skin of the patient so as to lift the skin and underlying tissue above the neighboring skin as to form a convex protrusion. The underlying tissue may contain one or more tumors that are to be pulverized by clamping, which is performed at the site of the protruded skin as shown. The clamping may take place for a duration of, e.g., from 1 to 3 seconds.

FIG. 4 is a table that lists various non-limiting examples of immune cells, such as dendritic cells, macrophages, and Langerhans cells, that may be activated in response to the methods of the invention. FIG. 4 additionally provides the location, major cell surface biomarkers, and functional contributions to the immune response for each cell type (reproduced from Nestle et al. Nat. Rev. Immunol. 9:679-691 (2009)).

FIG. 5 is a set of fluorescence micrographs showing uncompressed (left) and compressed (right) colonies of malignant breast epithelial cells. Malignant breast epithelial cells were cultured in a gelatinous substance that had been injected into flexible silicone chambers. The flexible chambers applied a compressive force to the cells at an early stage of development. Over time, the compressed malignant cells grew into more organized, healthy-looking acini that resembled normal structures, while malignant cells that were not compressed exhibited an asymmetric morphology consistent with uncontrolled cell growth. Time-lapse microscopy over several days showed that early compression also induced coherent rotation in the malignant cells, a characteristic feature of normal development (Cheng et al. PLoS ONE 4:e4632 (2009), the disclosure of which is incorporated herein by reference).

FIG. 6 is a diagram showing exemplary meridian lines of a human subject. A ventral (left) and dorsal (right) view of the meridian system, including the wood phase meridian, first fire phase meridian, second fire phase meridian, earth phase meridian, metal phase meridian, water phase meridian, lung meridian, heart meridian, liver meridian, spleen meridian, kidney meridian, pericardium meridian, large intestine meridian, small intestine meridian, stomach meridian, bladder meridian, and gall bladder meridian.

FIG. 7 is a table listing various patients that have undergone clamping therapy in order to treat a particular cancer. The table lists the age of each patient, as well as the type of cancer that was treated, the year the diagnosis of the cancer was rendered, when clamping therapy began, the duration of the therapy, and the total treatment period.

FIG. 8 is a table listing various patients that have undergone clamping therapy in order to treat additional diseases, including non-cancerous indications. The table lists the age of each patient, as well as the type of cancer that was treated, the year the diagnosis of the cancer was rendered, when clamping therapy began, the duration of the therapy, and the total treatment period.

### DETAILED DESCRIPTION

The invention provides methods of treating diseases, such as cancers, autoimmune diseases, infectious diseases, and diseases associated with major organs (e.g., kidney disease, renal disease, lung disease, and others) by clamping of the skin and underlying tissue of a subject (e.g., a human subject) in order to induce damage to the skin and/or tissue. The damage may manifest in pain or bruising of the subject at the site of the clamping. As such, the invention additionally encompasses kits containing clamps that are useful for the safe application of pressure to a patient by virtue of a regulator screw or similar adjustment mechanism capable of modulating the distance between the arms of a clamp used for clamping therapy.

The methods of the invention include procedures for the safe and efficacious clamping of a patient's skin to treat a variety of disorders, as well as protocols for activating the patient's immune system. In this way, clamping therapy harnesses the patient's natural immune defense mechanisms to eradicate, e.g., cancer cells or foreign microbial agents. The physiological events that underlie immune system activation include the induction of pain, e.g., by stimulating nociceptors at the epidermal surface that propagate action potentials through the central nervous system. The pain elicited by clamping promotes inflammation and stimulates immune system activity, which includes various immunological events, such as arteriole dilation and the migration of leukocytes into areas of damaged tissue. The clamping also induces the release of cell debris, such as uric acid, or salts thereof, such as monosodium urate, which further modulate immune activity.

Methods of the invention also include the use of skin clamping to apply pressure to, and in some instances to pulverize, cancerous lumps, such as solid tumors (e.g., breast epithelial tumors). The physical dissociation of these tissues induced by clamping serves multiple purposes, including promoting enhanced cell surface expression of tumor-associated antigens. These antigens can subsequently be recognized and eventually killed by cells of the immune system, such as CD4+ or CD8+ T-cells bearing a T-cell receptor that specifically binds such antigens. The beneficial effects of skin clamping are not limited to cancer, as a variety of additional disorders can be treated by clamping therapy and the enhanced immune activity thereby induced.

The sections that follow provide a description of how the skin clamping regimen may be performed, as well as a description of disorders that can be treated using skin clamping therapy.

### Clamping Protocol

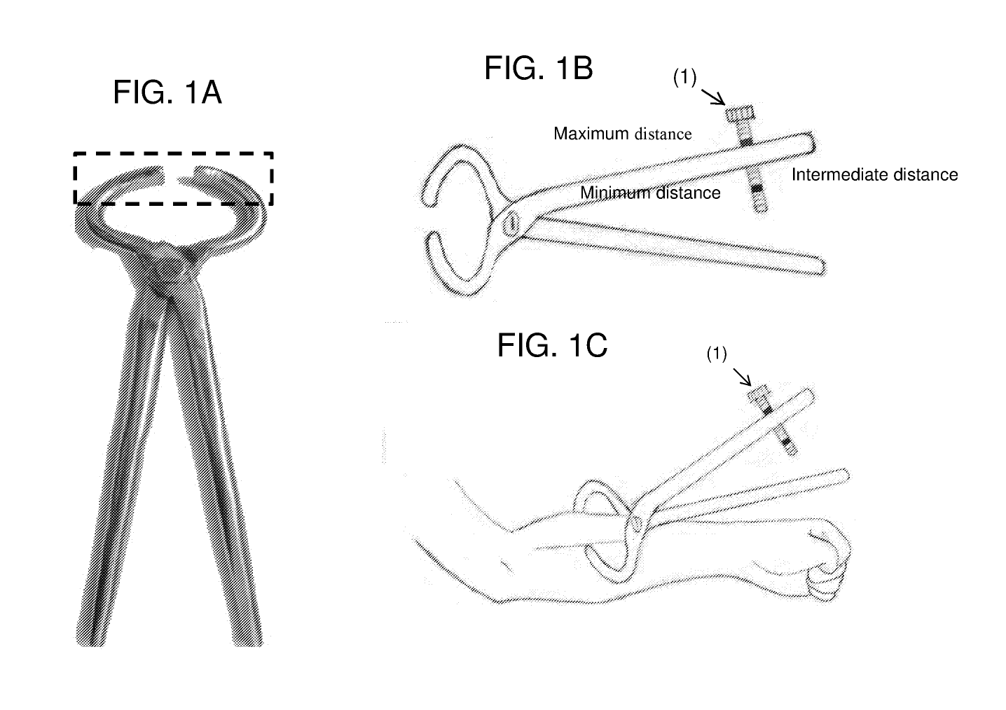

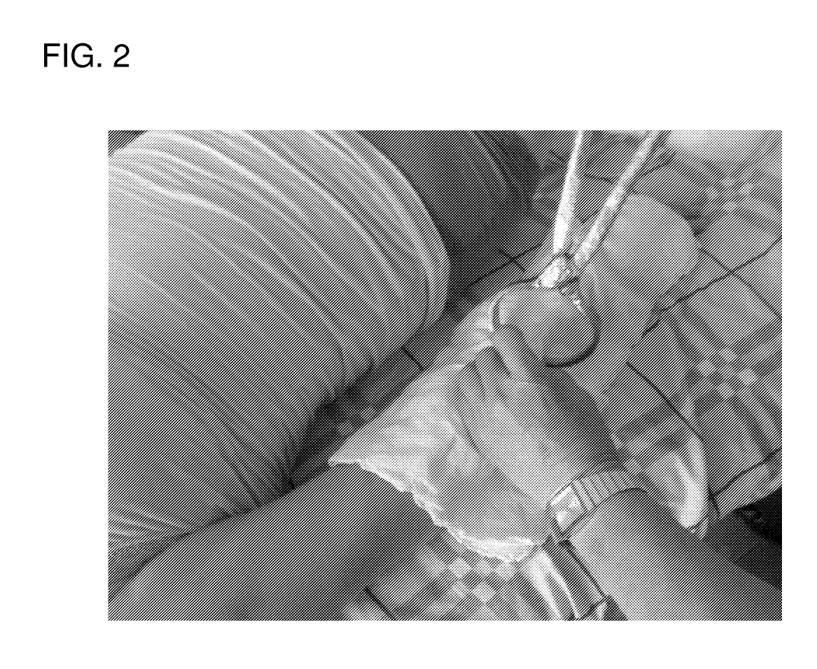

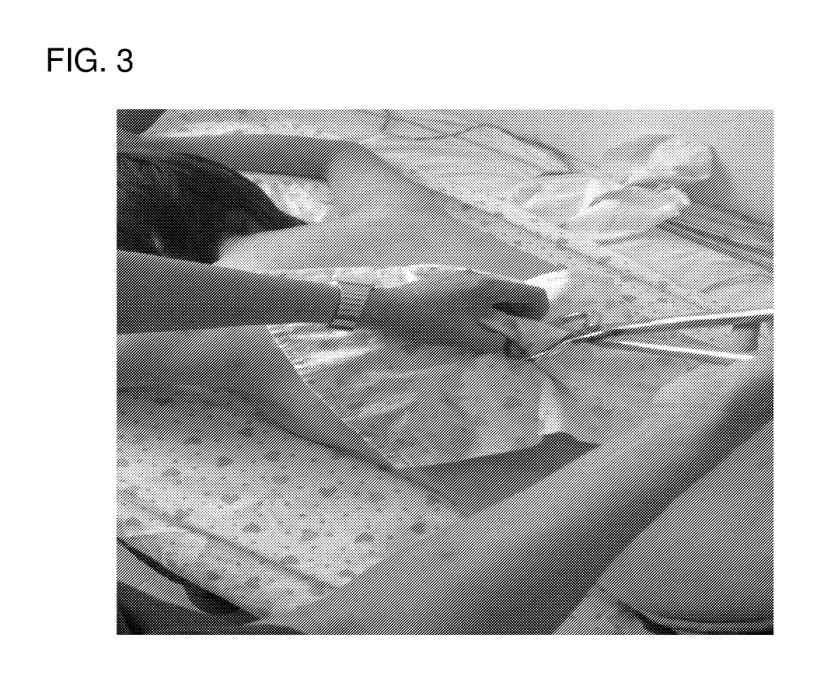

The methods of the invention can be used to treat a patient suffering from cancer, an autoimmune disease, an infectious disease, or another condition described herein. In some embodiments, the skin of the patient, as well as underlying tissue and/or muscle, are folded, e.g., in a convex protrusion of the skin that is lifted above the neighboring skin in a manner shown in any one of FIGS. 10, 2, and 3. A clamp, such as that shown in FIG. 1A or 1B, is then used to apply pressure to the base of the folded skin. This results in damage to the skin and/or the underlying tissue and/or muscle, and may also induce a sharp momentary pain. This clamping activity may also induce bruising at the site at which pressure is applied to the skin. Typically, patients are more sensitive to the pain in the first treatment and become tolerant in subsequent visits.

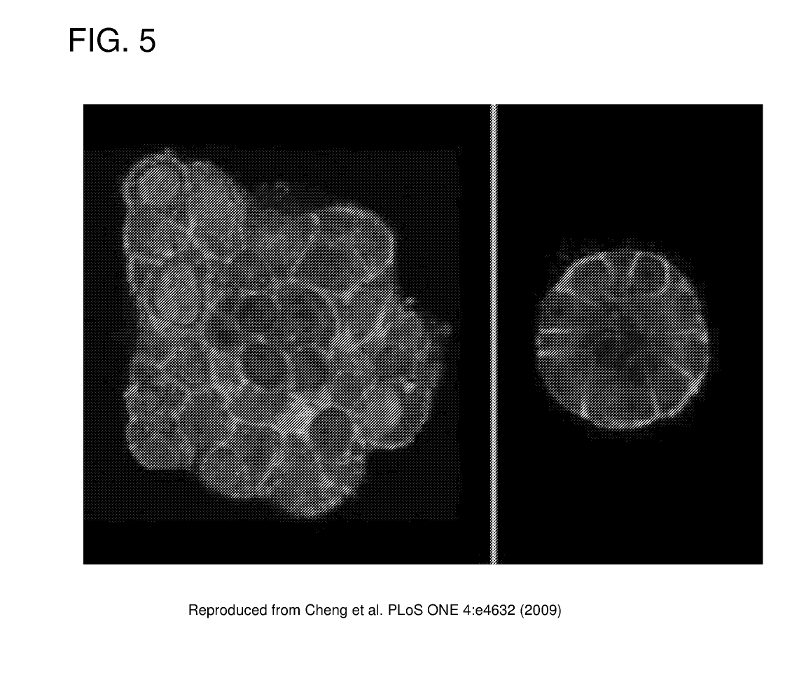

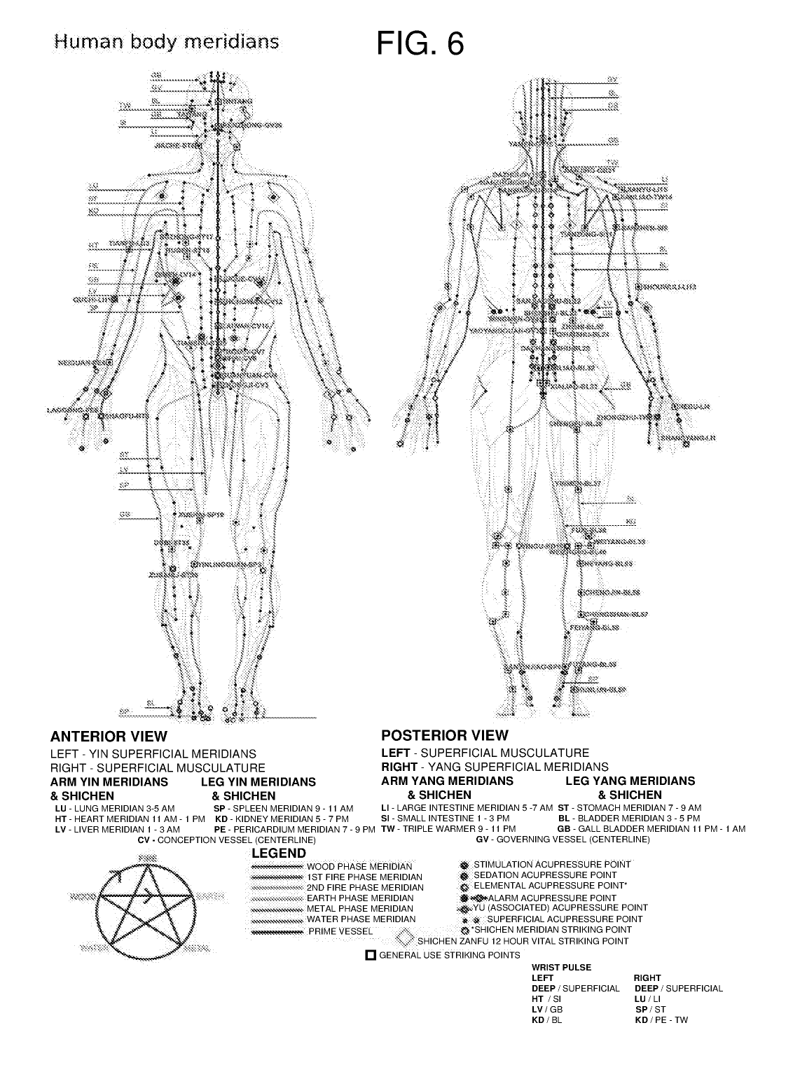

The patient may receive skin clamping therapy at the site of a particular disease, such as at the site of a solid tumor, or the patient may be administered clamping therapy along the entire body, e.g., in a manner that follows the meridian system on the ventral and dorsal sides of the body and limbs as described in FIG. 5.

As clamping therapy continues, often within month of treatment, lumps in various sizes (e.g., from 1-3 cm, or more, such as 1.0 cm, 1.1 cm, 1.2 cm, 1.3 cm, 1.4 cm, 1.5 cm, 1.6 cm, 1.7 cm, 1.8 cm, 1.9 cm, 2.0 cm, 2.1 cm, 2.2 cm, 2.3 cm, 2.4 cm, 2.5 cm, 2.6 cm, 2.7 cm, 2.8 cm, 2.9 cm, 3.0 cm, or more) rise to the surface of the skin. The lumps contain primarily uric acid or salts thereof, such as monosodium urate. Uric acid is frequently released as debris of dead cells that have been physically damaged due to the skin clamping procedure. The uric acid may that is released into peripheral tissues by the clamping of these lumps provides an additional signal that can stimulate immune cell activity. The intense initial pain induced by clamping therapy serves as another signal to stimulate the immune system. One or both of these phenotypes may occur concurrently with tissue inflammation, which is associated with a series of events that further potentiates the activity of immune cells, such as macrophages, dendritic cells, and T-cells. Together, these events may synergistically direct immune system resources to, e.g., malignant tissue or infectious microbial agents so as to kill, and ultimately eradicate, these cells.

A non-limiting example of a clamping procedure that can be used in conjunction with the methods of the invention is provided below:
1. Initially clamping can be performed from head to feet, or at one or more regions thereof, every day for 6 days, and then every other day for 16 times for a total of nearly 6 weeks. This constitutes one standard course of treatment. In general, for early stage cancer, seven or eight courses may be sufficient to achieve complete remission. During the later courses, the frequency of treatment can be reduced to once every 3 or 4 days, sometimes with a rest period of from about 2 to about 3 weeks between treatments.
2. There are six meridian lines located on the anterior (Yin) and posterior (Yan) sides of the human body. There are also meridian lines on the limbs, including three meridian lines on the anterior side of the arms (Yin) and three on posterior side (Yan), as well as three lines on the anterior side of legs

(Yin) and three on the posterior side (Yan). The clamping sites may be from about 2 to about 3 inches apart (e.g., from 2.0 inches, 2.1 inches, 2.2 inches, 2.3 inches, 2.4 inches, 2.5 inches, 2.6 inches, 2.7 inches, 2.8 inches, 2.9 inches, or 3.0 inches apart) along these meridian lines.
3. The skin, often together with underlying tissue, such as muscle tissue, is lifted by a physician of skill in the art. In some embodiments, the physician will use the left hand. The physician may then apply pressure to the base of the folded skin by pinching the skin between the physician's fingers. The clamp, held in the physician's free hand (e.g., the physician's right hand), is applied to the site at the base of the folded skin with the proper amount of force for from about 1 to about 3 seconds (e.g., 1 second) to produce a momentary sharp pain. This clamping activity may cause connective tissue at the base of the folded skin to break and physically dissociate from the bone surface. In this way, the connective tissue is separated from a physical substrate and can regenerate. For example, the rib bones of many cancer patients were found to be covered with white matter which can deform the rib cage and block the meridian system. The white matters are secreted from dead white cells. Skin clamping therapy is capable of physically breaking up this white matter and loosening it from the rib bones, thus allowing connective tissue to regenerate.
4. If desired or medically indicated, the connective tissue between muscles may be clamped intentionally to cause damage to old connective tissue and provide an opportunity for regeneration.
5. Excessive force can be avoided during the initial treatment, especially for patients that have undergone surgery or chemotherapy or that have heart problems. The areas of neck and upper breast can additionally be avoided in early stages of treatment.
6. In some embodiments, on the arm of the clamper (e.g., as shown in FIG. 1B), there is a regulator screw marked with three colors that indicate the closure distance at the head of the clamper. In some embodiments, one indicator, such as a color indicator (e.g., green), designates a large distance between the arms and is useful for initial stages of treatment so as to avoid applying excessive pressure. Another indicator, such as a color indicator (e.g., yellow), may designate an intermediate distance between the arms that is less than the distance used in the initial stages of treatment. This intermediate distance may be used for more advanced stages of therapy, e.g., once patients have developed a tolerance to the pain induced by clamping. A third indicator, such as a color indicator (e.g., red), may indicate a minimum distance between the arms of the clamp. This setting may be used to apply a greater amount of pressure than the previous settings, and may desirably be used during later stages of treatment.
7. As the body develops tolerance to pain, the clamping force may be increased. This will often produce lumps of various sizes that rise to the surface of the skin. In some embodiments, these lumps contain crystals of uric acid formed by dead cells. These lumps need not be removed surgically. Further clamping of lumps can often produce various sounds that resemble the breaking a glass ball. The injured skin of the patient may show a dark color associated bruising at the clamping site. After a few days, the dark color may fade away. In some embodiments, there is no pain experienced during further clamping. Clamping at this stage of therapy may lead to bleeding, which provides a mechanism of removing waste from the patient's damaged tissues.
8. In some embodiments, immediately after clamping a hair dryer may be used to blow warm air on the clamping site. The clamping site may also be touched with the hand so as to reduce pain and facilitate blood circulation for rapid healing of the injured skin.

### Cancers

Clamping therapy as described herein can be used to treat a variety of cancers, including those that manifest in solid tumors. In some instances, it may be desirable to perform clamping therapy at the sites of these solid tumors so as to physically damage the tumor. This clamping may cause an increase in antigen expression on the tumor surface, and may thus result in enhanced binding and activation of immune cells (e.g., antigen-presenting cells, such as dendritic cells and/or macrophages, as well as CD4+ and/or CD8+ T-cells) at the surfaces of cancerous cells therein.

In some instances, it may be desirable for a physician of skill in the art to monitor the efficacy of therapy by withdrawing a sample from the patient in order to analyze, e.g., the quantity of CD4+ T-cells, CD8+ T-cells, dendritic cells, and/or activated dendritic cells within the sample of the patient. A finding that the quantity of tumor-reactive CD4+ and/or CD8+ T-cells, or dendritic cells or activated dendritic cells, has increased relative to the quantity of the same cell type within a sample withdrawn from the patient prior to clamping therapy indicates that the therapy is efficacious. Additionally or alternatively, a physician may analyze the quantity of cancerous cells within a sample from the patient e.g., using flow cytometry or florescence-activated cell sorting (FACS) techniques known in the art. A finding that the quantity of cancerous cells in a sample isolated from the patient following clamping therapy has decreased relative to the quantity of the same cancerous cell type within a sample isolated from the patient prior to clamping therapy indicates that the clamping therapy is efficacious. In either case, a finding that the clamping therapy is not efficacious may indicate that subsequent clamping therapy is required, and/or that the intensity of the clamping is to be increased in future therapy (e.g., by increasing the amount of pressure applied by clamping, such as by adjusting a regulator screw so as to decrease the distance between the arms of the clamp used for the clamping therapy).

Cancers that can be treated according to the methods of the invention include, without limitation, leukemia, lymphoma, liver cancer, bone cancer, skin cancer, pulmonary cancer, brain cancer, bladder cancer, gastrointestinal cancer, breast cancer (e.g., late stage breast cancer, bilateral breast ductal carcinoma, or invasive bilateral breast cancer), cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, adenocarcinoma, pituitary adenoma, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrioma, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), ovarian carcinoma, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, uterine sarcoma, vaginal cancer, vulvar cancer, and waldenström macroglobulinemia.

In addition to the above indications, clamping therapy can be used to treat neoplasms, such as those characterized by a growth of the pancreas, salivary gland, pituitary gland, kidney, heart, lung, hematopoietic system, cranial nerves, heart, aorta, olfactory gland, hypopharynx, ear, nerves, structures of the head, eye, thymus, tongue, bone, liver, small intestine, large intestine, gut, brain, skin, peripheral nervous system, central nervous system, spinal cord, breast, embryonic structures, embryos, or testes, among others.

### Autoimmune Diseases

In addition to cancers and neoplasms, clamping therapy as described herein can be used to treat a variety of autoimmune diseases. For instance, a physician of skill in the art may administer clamping therapy to a patient suffering from one or more infectious diseases described herein. During the course of treatment, a physician may monitor the efficacy of therapy by withdrawing a sample from the patient in order to analyze, e.g., the quantity of self-reactive CD4+, CD8+ T-cells, dendritic cells, and/or activated dendritic cells within the sample of the patient. A finding that the quantity of self-reactive CD4+ and/or CD8+ T-cells, or dendritic cells or activated dendritic cells, has decreased relative to the quantity of the same cell type within a sample withdrawn from the patient prior to clamping therapy indicates that the therapy is efficacious. A finding that the clamping therapy is not efficacious (e.g., a finding that the quantity of self-reactive CD4+ and/or CD8+ T-cells, dendritic cells, or activated dendritic cells has increased) may indicate that subsequent clamping therapy may be needed, and/or that the intensity of the clamping may need to be increased in future therapy (e.g., by increasing the amount of pressure applied by clamping, such as by adjusting a regulator screw so as to decrease the distance between the arms of the clamp used for the clamping therapy).

Autoimmune diseases that can be treated using the methods of the invention include, without limitation, type I diabetes, alopecia areata, ankylosing spondylitis, antiphospholipid syndrome, autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, crest syndrome, cold agglutinin disease, Crohn's Disease, essential mixed cryoglobulinemia, fibromyalgia-fibromyositis, Graves' Disease, Guillain-Barré Syndrome, Hashimoto's thyroiditis, hypothyroidism, idiopathic pulmonary fibrosis, idiopathic thrombocytopenia purpura (ITP), IgA nephropathy, juvenile arthritis, lichen planus, lupus, systemic lupus erythematosus, Ménière's Disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, stiff-man syndrome, Takayasu Arteritis, temporal arteritis/giant cell arteritis, ulcerative colitis, uveitis, vasculitis, vitiligo, and Wegener's Granulomatosis.

### Infectious Diseases

In addition to the above, clamping therapy as described herein can be used to treat a variety of infectious diseases. For instance, a physician of skill in the art may administer clamping therapy to a patient suffering from one or more infectious diseases described herein. During the course of treatment, a physician may monitor the efficacy of therapy by withdrawing a sample from the patient in order to analyze, e.g., the quantity of CD4+ T-cells, CD8+ T-cells, dendritic cells, and/or activated dendritic cells within the sample of the patient. A finding that the quantity of CD4+ T-cells, CD8+ T-cells, dendritic cells, or activated dendritic cells has increased relative to the quantity of the same cell type within a sample withdrawn from the patient prior to clamping therapy indicates that the therapy is efficacious. Additionally or alternatively, a physician may analyze the quantity of DNA within a sample from the patient that is of bacterial, viral, or fungal origin, e.g., using quantitative polymerase chain reaction techniques known in the art (e.g., quantitative RT-PCR). A finding that the quantity of DNA of bacterial, viral, or fungal origin in a sample isolated from the patient following clamping therapy has decreased relative to the quantity of the same DNA molecule within a sample isolated from the patient prior to clamping therapy indicates that the clamping therapy is efficacious. In either case, a finding that the clamping therapy is not efficacious may indicate that subsequent clamping therapy may be needed, and/or that the intensity of the clamping may need to be increased in future therapy (e.g., by increasing the amount of pressure applied by clamping, such as by adjusting a regulator screw so as to decrease the distance between the arms of the clamp used for the clamping therapy).

Infectious diseases that can be treated using the methods of the invention include, without limitation, an infectious disease caused by virus, a bacterium, a fungus, or a parasite.

In some embodiments, the virus is selected from the group consisting of Gadgets Gully virus, Kadam virus, Kyasanur Forest disease virus, Langat virus, Omsk hemorrhagic fever virus, Powassan virus, Royal Farm virus, tick-borne encephalitis virus, Louping ill virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, and Rio Bravo virus, Venezuelan equine encephalitis virus (VEE), Eastern equine encephalitis virus (EEE), Western equine encephalitis virus (WEE), Ebola virus, Marburg virus, smallpox virus, vaccinia virus, Lassa virus, Ippy virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, and Whitewater Arroyo virus, Sin Nombre virus, Hantaan virus, Rift Valley fever virus, Crimean-Congo hemorrhagic fever virus, Dugbe virus, herpes simplex virus (HSV), cytomegalovirus (CMV), Epstein-Barr virus (EBV), Kaposi's sarcoma associated-herpesvirus (KSHV), influenzavirus A, H5N1 avian influenza virus, influenzavirus B, influenzavirus C, severe acute respiratory syndrome (SARS) virus, rabies virus, and vesicular stomatitis virus (VSV).

In some instances, the bacterium is selected from the group consisting of Pseudomonas aeruginosa, Salmonella typhimurium, Escherichia coli, Klebsiella pneumoniae, Bruscella, Burkholderia mallei, Yersinia pestis, and Bacillus anthracis.

The fungus may be selected from the group consisting of Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, and Rhizopus arrhizus.

In some embodiments, the parasite is selected from the group consisting of Toxoplasma gondii, Plasmodium falciparum, P. vivax, P. ovale, P. malariae, Trypanosoma spp., and Legionella spp.

### Additional Indications

In addition to the diseases described above, clamping therapy may additionally be used to treat a variety of other diseases. These indications include, without limitation, hypertension, hyperglycemia, hyperlipidemia, edema, obesity, infertility, amenorrhea, fatigue, vertigo, uterine bleeding, uterine ulcer, hyperthyroidism, myoma, endometriosis, cerebral palsy, brain atrophy, systemic muscular atrophy, trigeminal neuralgia, schizophrenia, epilepsy, amyotrophic lateral sclerosis (ALS), Parkinson's Disease, autism, Alzheimer's Disease, Huntington's Disease, emphysema, asthma, hepatitis B, cough, systemic fibroma, renal diseases, lung diseases, and liver diseases.

### Immune System Activation

### Multiple Roles of the Epidermal Surface

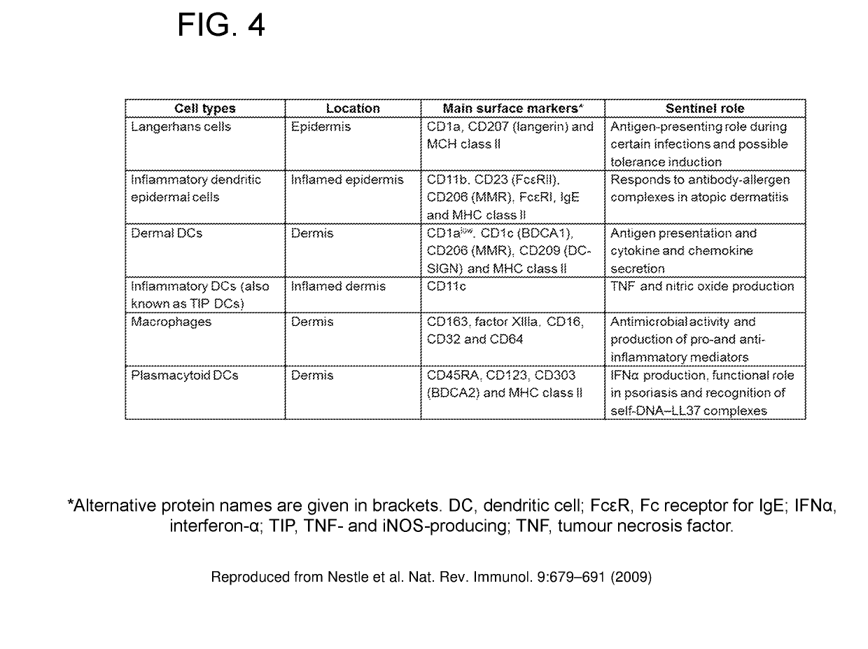

The methods of the invention provide a strategy for stimulating the immune system in response to tissue damage and physical pain. Human skin, as the first line of defense against bodily insults and microbial pathogens, provide protection to the human body from injury and infection. The skin provides this protection by orchestrating the activities of various cell populations. Keratinocytes exert an important influence over both innate and adaptive responses, as these cells potentiate the early phase of the innate immune response to invading pathogens by promoting the release of chemokines and cytokines, thus attracting key effector immune cells to the site of the foreign pathogen. These cells also stimulate antigen-experienced T cells during inflammation. In addition, spatially and functionally distinct skin-resident dendritic cell populations serve as important immune sentinels, as summarized in FIG. 4. Dendritic cells (DCs) are white blood cells that direct various immune responses. The most potent antigen presenting cells known, they modulate host immunity and sense microbes through the use of pathogen recognition receptors. The skin contains multiple phenotypically and spatially distinct DC populations, including Langerhans cells in the epidermis and DCs in the dermis. The presence of these distinct populations promotes the development and control of skin immune responses. The high quantities of accessible DCs in the skin relative to other vaccination sites have rendered the skin an excellent target for immunotherapy. Accumulating data have shown that tissue-resident rather than circulating T cells play a key role in skin homoeostasis and pathology, as evidenced by the fact that normal skin contains more than twice as many T cells as the blood, and 98% of CLA+ skin-homing lymphocytes reside in the skin under normal physiological conditions.

Apart from acting as a barrier to protect the body against invading pathogens, the skin also functions as a sensory interface, containing abundant sensory fibers. Interestingly, a synergy between temperature-sensory and pain-sensory fibers and immune cells is plausible for several reasons. For instance, these fibers are present in all types of tissue communicating with the outside. Additionally, tissue damage, which requires immune responses, often occurs together with pain stimuli. Pain sensory fibers can directly react to immune-relevant stimuli by expressing danger and damage receptors. These fibers can also conduct signals not only from periphery to central nervous system (CNS) but also from the CNS to the periphery, promoting the release of neural mediators that can act locally on immune cell. Pain sensory neurons are also able to generate local immune responses, such as through the propagation of action potentials stimulated by the opening of TRP channels, such as TRPVI.

When the skin is clamped, it induces skin damage (e.g., physical injury) that manifests as a red area, which provides a sign of inflammation. This is because the arterioles of the injured skin have dilated and the capillaries have filled with blood and become more permeable, allowing fluid and blood proteins to move into the space between tissues. Inflammation is the body's attempt at self-protection and removal of harmful stimuli, including damaged cells, irritants or pathogens, and at self-protection and removal of harmful stimuli, including damaged cells, irritants or pathogens, and initiation of the healing process. Within a few minutes after skin clamping, the clamped tissue is injured and acute inflammation starts to occur. Three main processes occur during acute inflammation: (1) arterioles (small branches of arteries that lead to capillaries that supply blood to the damaged region) dilate, resulting in increased blood flow; (2) capillaries become more permeable, so fluid and blood proteins can move into interstitial spaces; and (3) neutrophils and possibly some macrophages migrate out of capillaries and venules (small veins that go from a capillary to a vein) and move into interstitial spaces.

### Uric Acid Release

After a few months of treatment, it was found that solid lumps or nodules (1-3 cm in diameter) rise to the surface of the skin. As part of the skin clamping treatment protocol, these lumps were crushed, like breaking a glass ball. Pathological examination revealed the material to be uric acid crystals, that is, monosodium urate (MSU). As a ubiquitous metabolite it is produced in high quantities upon cellular injury, uric acid generates effects that may be considerable in health and disease. Uric acid—in the form of monosodium urate crystals precipitated in synovial and cavities and other anatomic location—is a well-known cause of gout, inducing symptoms such as strong inflammation and debilitating pain. In the recent years, more and more research studies have shown that uric acid crystal deposition in tissues, identified as an endogenous adjuvant that drives immune responses in the absence of microbial stimulation, is a strong inflammatory stimulant. This simple crystalline structure appears to activate innate host defense mechanisms in multiple ways and trigger robust inflammation. The recognition mechanisms of MSU following its phase change from soluble uric acid are diverse, involving protein receptors and non-specific plasma membrane attachment. Upon contact with host cells, MSU induces a set of membrane events that trigger Syk and PI3K activation, phagocytosis, and cytokine production. Having entered the cell, MSU further triggers NALP3 inflammasome activation and induces the production of IL-1β, likely inducing a full spectrum of inflammation and immune response.

Uric acid's inflammatory effects involve its precipitation into MSU crystals, which are further recognized by innate phagocytes such as dendritic cells, macrophages and neutrophils. During the recognition process, antigen-presenting cells can sense uric acid as one of the proinflammatory endogenous signals released by damaged tissues and cells. Noticeably, these damage-associated signals can trigger a systemic inflammatory response similar to pathogen-associated molecular patterns. In addition, uric acid stimulates a type of immune cell (dendritic cell, DC) to mature. When DCs are injected along with certain substances in experimental animals, another type of immune cell, CD8+ T cell, becomes stronger in its response to microbes and noxious chemicals. In other experiments, removal of uric acid was shown to weaken certain types of immune responses, indicating an immune-boosting effect of uric acid. Interestingly, DCs are important for host immunity, and sense microbes with pathogen recognition receptors. New evidence indicates that DCs also sense uric acid crystals in dead cells, suggesting that the immune system is conscious not only of pathogens but also of death and danger. In another words, uric acid is a principal endogenous danger signal released from dead and dying cells. It is a breakdown product of nucleic acids such as DNA in response to cell damage. Whenever a large number of cells die, for example when a tumor is being medically treated or during an infection, the uric acid becomes more concentrated and the molecules crystallize. Dead cells not only release intracellular stores of uric acid, but also produced it in large amounts postmortem as nucleic acids are degraded.

### Combination Therapy

The skin clamping methods described herein can be used alone or in conjunction with additional techniques to treat or prevent cancers or diseases, such as infectious diseases, in a subject. For instance, a physician of skill in the art can administer skin clamping therapy to a subject suffering from cancer, such as a cancer described herein, in order to stimulate an immune response within the subject against endogenous cancer cells. The subject may also be administered an additional anti-cancer therapeutic, such as a CAR T treatment in which, e.g., autologous, allogeneic, or syngeneic T-cells are genetically re-engineered so as to express a T-cell receptor protein that specifically binds a tumor-associated antigen in order to localize the T-cell (e.g., a CD8+ cytotoxic T-cell) to the surface of the cancer cell and initiate an immune response. Other adjunctive therapies known in the art for treating a diagnosed disease condition, e.g., cancer and infectious disease, can be administered in combination with the clamping therapy to provide a holistic treatment regimen.

Whether administered alone or in conjunction with additional therapeutic agents, the skin clamping techniques described herein are advantageous in that these methods represent a comprehensive immunotherapy that functions by activating a full array of endogenous immune system activities in order to alleviate a broad spectrum of disease, including cancers and other disorders, such as infectious diseases. Moreover, the skin clamping techniques described herein would be expected to be significantly less expensive than other pharmaceutical-based therapies, such as anti-cancer therapies, that are currently used or being developed, and thus provide a robust and accessible therapeutic modality for the treatment of a wide range of diseases.

### Kits

The invention additionally provides kits containing, e.g., a clamp that can be used in conjunction with any of the methods of the invention described herein. An exemplary clamp is shown in FIG. 1B. This clamp contains a regulator screw that can be used to modulate the pressure that is applied to a patient by regulating the distance between the arms of the clamp. This serves to regulate the distance between the ends of the clamp when applied to the skin of a patient.

The regulator screw can be adjusted such that the portion of the screw that is situated between the two arms of the clamp is longer (e.g., such that the distance between the two arms of the clamp is maximized as shown in FIG. 1B), shorter (e.g., the distance between the two arms of the clamp is minimized as shown in FIG. 1B), or intermediate (e.g., such that the distance between the arms of the clamp is in between the distances of the previous two settings, for instance, as shown in FIG. 1B) so as to modulate the distance between the arms of the clamp when the clamp is in a fully-closed state. In this way, a physician of skill in the art can regulate the amount of pressure delivered to a patient. For instance, a physician may deliver less pressure during initial stages of treatment by clamping the skin and underlying tissue of a patient using a clamp that is maintained in a state of maximum distance between the arms, e.g., by adjusting the regulator screw such that the distance between the two arms of the clamp is maximized. A physician may gradually decrease the distance between the arms of the clamp as the therapy regimen continues, e.g., by adjusting the regulator screw such that the distance between the two arms is less than the previous setting. As treatment progresses, a physician may adjust the regulator screw, e.g., such that the distance between the two arms of the clamp is minimized. These directives may be provided within a package insert contained within a kit of the invention. Regulation of the distance between the arms of the clamp thus allows a user of the clamp to apply safe amounts of pressure to the skin and underlying tissue of a patient while maintaining therapeutic efficacy (e.g., reducing the quantity of cancerous cells within a patient, increasing the quantity of tumor-reactive immune cells, decreasing the quantity of autoreactive immune cells, and/or decreasing the quantity of pathogen-derived DNA as described above).

### EXAMPLES

The following examples are put forth so as to provide those of ordinary skill in the art with a description of how the methods and kits described herein may be used, made, and evaluated, and are intended to be purely exemplary of the invention and are not intended to limit the scope of what the inventors regard as their invention.

### Example 1

### Clamping Therapy as a Treatment Modality for Various Cancers

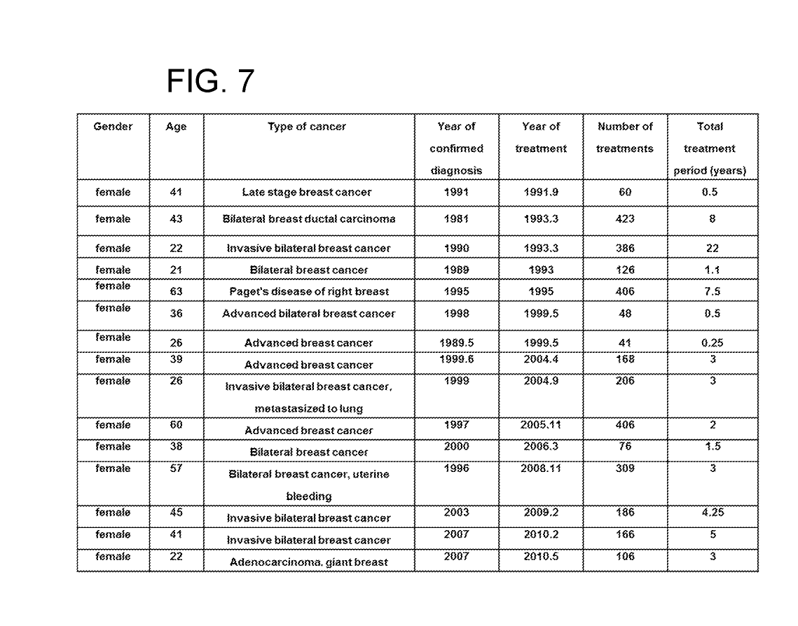

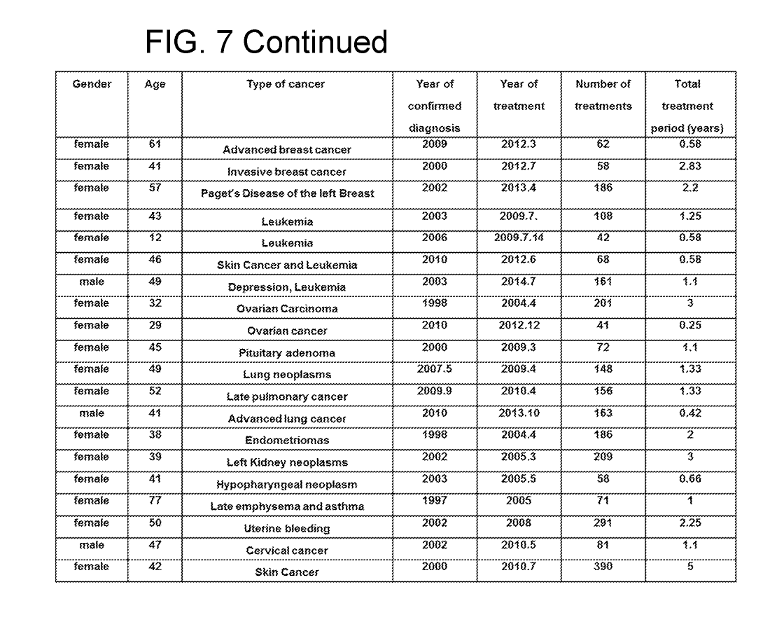

Clamping therapy as described herein can be used to treat a variety of cancers, including those that manifest with solid tumors as well as cancers of the blood. A group of patients that were previously diagnosed with a particular cancer were treated using clamping therapy protocols described herein. FIG. 7 provides a list of the patients that were treated using these procedures. As FIG. 7 demonstrates, a wide variety of cancers can be treated using clamping therapy, including various forms of breast cancers and blood cancers. Clamping therapy typically occurred over the course of several years. Remission of the cancer was observed in each of these patients by the conclusion of the treatment period shown in the right column of FIG. 7.

### Example 2

### Clamping Therapy Can Be Used to Treat Non-cancerous Indications

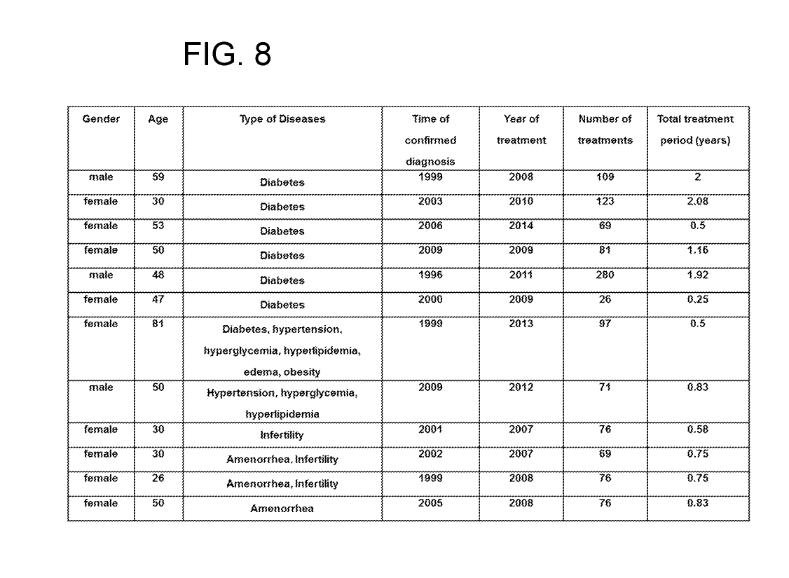

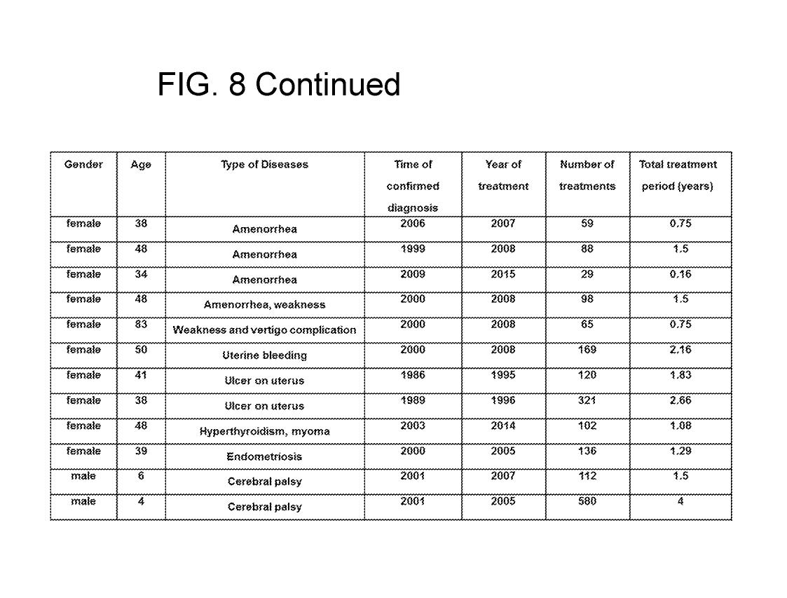

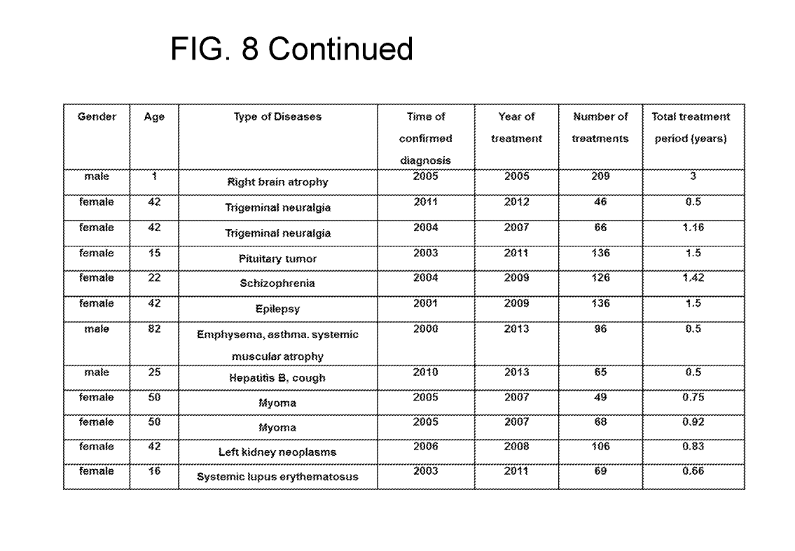

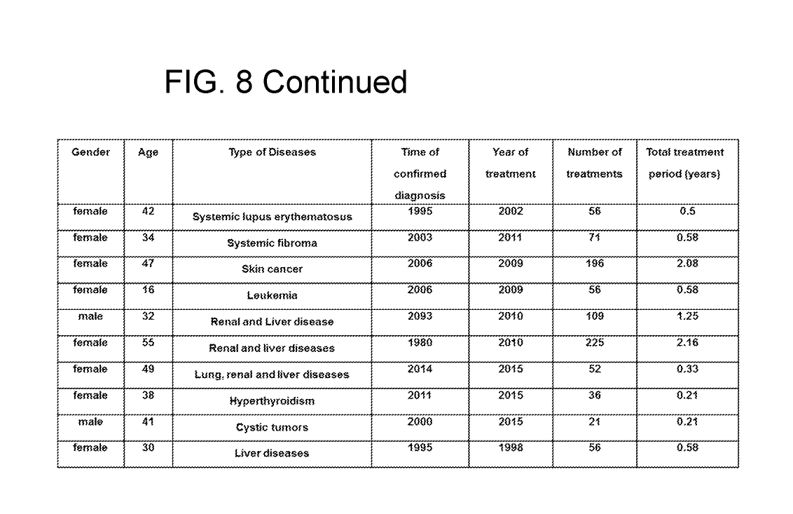

In addition to cancers, clamping therapy described herein can also be used to treat a variety of non-cancerous diseases. As shown in FIG. 8, a variety of patients presenting with diseases including diabetes, organ conditions, depression, and brain atrophy were treated using the skin clamping techniques described herein. Treatment durations varied from less than one year to greater than two years depending on the condition being treated and the individual patient. In all cases, the patients were previously diagnosed as having the indicated disorder. All patients demonstrated an improvement in their physical condition at the conclusion of the treatment period shown in the right column of FIG. 8.

### Example 3

### Administration of Clamping Therapy to Treat Breast Cancer

A physician of skill in the art can administer clamping therapy as described herein to a patient that has been diagnosed with breast cancer. For instance, the physician may use a clamp, such as a clamp described herein containing a regulator screw, in order to administer clamping therapy at the site of the breast tumor and/or along meridian lines throughout the patient's entire body. A physician may begin the treatment regimen by administering lower quantities of pressure, e.g., by using a clamp that is maintained in a state of elevated distance between the arms of the clamp. As the treatment progresses (e.g., after several days, weeks, or months), solid lumps may rise to the epidermal surface of the patient. These lumps may contain uric acid crystals, or a salt thereof, such as monosodium urate (MSU). The presence of MSU can be determined using techniques known in the art, such as ¹H or ¹³C nuclear magnetic resonance spectroscopy (NMR). A physician may desirably administer clamping therapy at the sites of these MSU lumps in order to physically break the crystals therein and promote the release of MSU into peripheral tissues, which may in turn activate one or more dendritic cells.

In some instances, it may be desirable for a physician of skill in the art to monitor the efficacy of therapy by withdrawing a sample from the patient in order to analyze, e.g., the quantity of CD4+ T-cells, CD8+ T-cells, dendritic cells, or activated dendritic cells within the sample of the patient. A finding that the quantity of tumor-reactive CD4+ and/or CD8+ T-cells, or dendritic cells or activated dendritic cells has increased relative to the quantity of the same cell type within a sample withdrawn from the patient prior to clamping therapy indicates that the therapy is efficacious. Additionally or alternatively, a physician may analyze the quantity of cancerous cells within a sample from the patient e.g., using flow cytometry or florescence-activated cell sorting (FACS) techniques known in the art. A finding that the quantity of cancerous cells in a sample isolated from the patient following clamping therapy has decreased relative to the quantity of the same cancerous cell type within a sample isolated from the patient prior to clamping therapy indicates that the clamping therapy is efficacious. In either case, a finding that the clamping therapy is not efficacious may indicate that subsequent clamping therapy is required, and/or that the intensity of the clamping is to be increased in future therapy (e.g., by increasing the amount of pressure applied by clamping, such as by adjusting a regulator screw so as to decrease the distance between the arms of the clamp used for the clamping therapy).

### Example 4

### Administration of Clamping Therapy to Treat an Infectious Disease

A physician of skill in the art may administer clamping therapy to a patient suffering from one or more infectious diseases described herein, such as a bacterial infection. A physician of skill in the art may administer clamping therapy at the site of the infection (e.g., at the skin that shows signs of infection, such as irritation, rash, redness, inflammation, or physical discomfort). In some cases, clamping therapy may be performed across meridian lines along all or a portion of the patient's body. The treatment may persist for several weeks, months, or years, and may be performed one or more times each day, every other day, every 3 days, every 4 days, every 5 days, every 6 days, every week, every 2 weeks, every 3 weeks, every 4 weeks, every month, every 2 months, every 3 months, or more. For instance, a treatment regimen may involve a physician treating a patient according to a standard treatment course as described herein (i.e., clamping therapy may be administered once daily for 6 days, followed by once every other day for 16 additional days to form a standard treatment course). When desirable, the standard treatment course may be repeated between 2 and 20 times, such as 7 or 8 times.

During the course of treatment, a physician may monitor the efficacy of therapy by withdrawing a sample from the patient in order to analyze, e.g., the quantity of CD4+ T-cells, CD8+ T-cells, dendritic cells, or activated dendritic cells within the sample of the patient. A finding that the quantity of CD4+ and/or CD8+ T-cells, or dendritic cells or activated dendritic cells has increased relative to the quantity of the same cell type within a sample withdrawn from the patient prior to clamping therapy indicates that the therapy is efficacious. Additionally or alternatively, a physician may analyze the quantity of DNA within a sample from the patient that is of bacterial origin, e.g., using quantitative polymerase chain reaction techniques known in the art (e.g., quantitative RT-PCR). A finding that the quantity of DNA of bacterial origin in a sample isolated from the patient following clamping therapy has decreased relative to the quantity of the same DNA molecule within a sample isolated from the patient prior to clamping therapy indicates that the clamping therapy is efficacious. In either case, a finding that the clamping therapy is not efficacious may indicate that subsequent clamping therapy may be needed, and/or that the intensity of the clamping may need to be increased in future therapy (e.g., by increasing the amount of pressure applied by clamping, such as by adjusting a regulator screw so as to decrease the distance between the arms of the clamp used for the clamping therapy).

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating a disease in a subject, said method comprising enclosing a portion of tissue comprising skin of said subject within a clamp and clamping the tissue for a duration and with sufficient pressure to cause damage to said tissue.

2. The method of claim 1, wherein the method comprises folding said portion to form a crease prior to said enclosing.

3. The method of claim 2, wherein the folding comprises lifting said skin to form a region of skin that is convex with respect to surrounding skin that is not folded.

4. The method of any one of claims 1-3, wherein the portion of tissue comprises one or more tumors.

5. The method of claim 4, wherein the clamping causes crushing of said one or more tumors.

6. The method of any one of claims 1-5, wherein the method further comprises compressing one or more muscles and/or connective tissues located underneath said skin.

7. The method of any one of claims 1-6, wherein the pressure is applied at a location at which said skin converges with surrounding skin that is not folded.

8. The method of any one of claims 1-7, wherein the clamp comprises curved ends and a flexible handle.

9. The method of claim 8, wherein the flexible handle is configured to allow a user of said clamp to control the duration of pressure applied by said clamping.

10. The method of claim 8 or 9, wherein the flexible handle is configured to allow a user of said clamp to control the amount of pressure applied by said clamping.

11. The method of any one of claims 1-10, wherein the clamp comprises a mechanism capable of regulating distance between said curved ends.

12. The method of any one of claims 1-11, wherein the clamp comprises arms and a regulator screw capable of maintaining a desired distance between the arms of the clamp.

13. The method of claim 12, wherein the regulator screw can be adjusted to one of a plurality of settings.

14. The method of claim 13, wherein the plurality of settings correspond to a state of maximum distance between the arms of the clamp, one or more states of intermediate distance between the arms of the clamp, and a state of minimum distance between the arms of the clamp.

15. The method of claim 14, wherein the maximum distance is between about 2.4 cm and about 7.2 cm.

16. The method of claim 14, wherein the intermediate distance is between about 1.6 cm and about 4.8 cm.

17. The method of claim 14, wherein the minimum distance is between about 0.8 and about 2.4 cm.

18. The method of any one of claims 14-17, wherein the setting that corresponds to the state of maximum distance between the arms of the clamp is indicated by exposure of a green color adjacent to an arm of the clamp.

19. The method of any one of claims 14-18, wherein the setting that corresponds to said state of intermediate distance between the arms of said clamp is indicated by exposure of a yellow color adjacent to an arm of said clamp.

20. The method of any one of claims 14-19, wherein the setting that corresponds to said state of minimum distance between the arms of said clamp is indicated by exposure of a red color adjacent to an arm of said clamp.

21. The method of any one of claims 1-20, wherein the clamping occurs for a period of about 1 to about 10 seconds.

22. The method of claim 21, wherein the clamping occurs for a period of about 1 to about 3 seconds.

23. The method of any one of claims 1-22, wherein the clamping is performed one or more times daily, weekly, monthly, or yearly.

24. The method of claim 23, wherein the clamping is performed one or more times daily.

25. The method of claim 24, wherein the clamping is performed one or more times daily for between about two and about twenty days.

26. The method of claim 25, wherein the clamping is performed one or more times daily for six days.

27. The method of claim 26, wherein at the end of said six days, the clamping is repeated one or more times every other day for a total of 16 additional days to form a standard treatment course.

28. The method of claim 27, wherein the standard treatment course is repeated between two and twenty times.

29. The method of claim 28, wherein the standard treatment course is repeated between five and fifteen times.

30. The method of claim 29, wherein the standard treatment course is repeated between six and nine times.

31. The method of claim 30, wherein the standard treatment course is repeated seven times.

32. The method of claim 30, wherein the standard treatment course is repeated eight times.

33. The method of any one of claims 27-32, wherein the clamping is performed with increasing pressure over the duration of said standard treatment course.

34. The method of any one of claims 1-33, wherein the clamping is sufficient to cause pain and/or bruising to said tissue.

35. The method of any one of claims 1-34, wherein the clamping causes an increase in the amount or concentration of one or more antigens on the surface of a cancer cell within said subject.

36. The method of any one of claims 1-35, wherein the clamping causes an increase in the amount or concentration of one or more dendritic cells on the surface of a cancer cell within said subject.

37. The method of claim 36, wherein the dendritic cells are located below said skin.

38. The method of any one of claims 1-37, wherein the subject exhibits an elevated immune response after said clamping is performed.

39. The method of claim 38, wherein the elevated immune response is selected from the group consisting of inflammation, secretion of chemokines, an increase in the level of one or more cytokines, and activation of one or more immune cells within said subject.

40. The method of claim 39, wherein the inflammation comprises one or more processes selected from the group consisting of arteriole dilation, an increase in capillary permeability, and migration of neutrophils and/or macrophages from capillaries or venules into interstitial spaces.

41. The method of claim 39, wherein the chemokines are capable of attracting to said portion of tissue one or more macrophages, T-cells, mast cells, dendritic cells, activated dendritic cells, eosinophils, and/or neutrophils.

42. The method of claim 39, wherein the one or more cytokines are selected from the group consisting of TNFα, IFNγ, IL-1, IL-6, and IL-8.

43. The method of claim 39, wherein the one or more immune cells are selected from the group consisting of macrophages, neutrophils, T-cells, antigen-presenting cells, and dendritic cells.

44. The method of any one of claims 1-43, wherein the subject exhibits epidermal lumps after said clamping is performed.

45. The method of claim 44, wherein the lumps are between about 1 cm and about 3 cm in length.

46. The method of claim 44 or 45, wherein the method further comprising the step of compressing said lumps.

47. The method of claim 46, wherein the lumps are broken into a plurality of fragments.

48. The method of claim 46 or 47, wherein the lumps comprise cell debris.

49. The method of claim 48, wherein the cell debris is or comprises monosodium urate.

50. The method of claim 49, wherein the monosodium urate is in a crystalline form.

51. The method of claim 49 or 50, wherein the monosodium urate is released within said subject.

52. The method of claim 51, wherein the monosodium urate is released into peripheral tissues within said subject.

53. The method of any one of claims 49-52, wherein the monosodium urate promotes the growth or maturation of one or more cells selected from the group consisting of macrophages, neutrophils, T-cells, antigen-presenting cells, and dendritic cells.

54. The method of any one of claims 49-53, wherein the monosodium urate induces the production of IL-1β in said subject.

55. The method of any one of claims 41, 43, and 53, wherein the T-cells are CD4+ or CD8+ T-cells.

56. The method of any one of claims 41, 43, 53, and 55, wherein the T-cells are capable of specifically binding an antigen expressed on the surface of a cancer cell.

57. The method of claim 56, wherein the T-cells are capable of killing one or more of said cancer cells.

58. The method of any one of claims 1-57, wherein the skin is located along a meridian line of said subject.

59. The method of claim 58, wherein the meridian line is selected from the group consisting of wood phase meridian, first fire phase meridian, second fire phase meridian, earth phase meridian, metal phase meridian, water phase meridian, lung meridian, heart meridian, liver meridian, spleen meridian, kidney meridian, pericardium meridian, large intestine meridian, small intestine meridian, stomach meridian, bladder meridian, and gall bladder meridian.

60. The method of any one of claims 1-59, wherein the disease is a cancer.

61. The method of claim 60, wherein the cancer is selected from the group consisting of leukemia, lymphoma, liver cancer, bone cancer, skin cancer, pulmonary cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, adenocarcinoma, pituitary adenoma, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrioma, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), ovarian carcinoma, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, uterine sarcoma, vaginal cancer, vulvar cancer, and waldenström macroglobulinemia.

62. The method of claim 60, wherein the cancer is selected from the group consisting of breast cancer, adenocarcinoma, leukemia, skin cancer, ovarian carcinoma, pituitary adenoma, pulmonary cancer, endometrioma, and cervical cancer.

63. The method of claim 62, wherein the breast cancer is selected from the group consisting of late stage breast cancer, bilateral breast ductal carcinoma, and invasive bilateral breast cancer.

64. The method of any one of claims 1-59, wherein the disease is a neoplasm.

65. The method of claim 64, wherein the neoplasm is a growth of a tissue or organ selected the group consisting of a pancreas, salivary gland, pituitary gland, kidney, heart, lung, hematopoietic system, cranial nerves, heart, aorta, olfactory gland, hypopharynx, ear, nerves, structures of the head, eye, thymus, tongue, bone, liver, small intestine, large intestine, gut, brain, skin, peripheral nervous system, central nervous system, spinal cord, breast, embryonic structures, embryos, and testes.

66. The method of claim 65, wherein the neoplasm is a growth of an organ selected from the group consisting of a lung, kidney, and hypopharynx.

67. The method of any one of claims 1-59, wherein the disease is an autoimmune disease selected from the group consisting of type I diabetes, alopecia areata, ankylosing spondylitis, antiphospholipid syndrome, autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, crest syndrome, cold agglutinin disease, Crohn's Disease, essential mixed cryoglobulinemia, fibromyalgia-fibromyositis, Graves' Disease, Guillain-Barré Syndrome, Hashimoto's thyroiditis, hypothyroidism, idiopathic pulmonary fibrosis, idiopathic thrombocytopenia purpura (ITP), IgA nephropathy, juvenile arthritis, lichen planus, lupus, systemic lupus erythematosus, Ménière's Disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, stiff-man syndrome, Takayasu Arteritis, temporal arteritis/giant cell arteritis, ulcerative colitis, uveitis, vasculitis, vitiligo, and Wegener's Granulomatosis.

68. The method of claim 67, wherein the autoimmune disease is selected from the group consisting of type I diabetes and lupus.

69. The method of any one of claims 1-59, wherein the disease is an infectious disease caused by one or more agents selected from the group consisting of a virus, a bacterium, a fungus, or a parasite.

70. The method of claim 69, wherein the virus is selected from the group consisting of Gadgets Gully virus, Kadam virus, Kyasanur Forest disease virus, Langat virus, Omsk hemorrhagic fever virus, Powassan virus, Royal Farm virus, tick-borne encephalitis virus, Louping ill virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, and Rio Bravo virus, Venezuelan equine encephalitis virus (VEE), Eastern equine encephalitis virus (EEE), Western equine encephalitis virus (WEE), Ebola virus, Marburg virus, smallpox virus, vaccinia virus, Lassa virus, Ippy virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, and Whitewater Arroyo virus, Sin Nombre virus, Hantaan virus, Rift Valley fever virus, Crimean-Congo hemorrhagic fever virus, Dugbe virus, herpes simplex virus (HSV), cytomegalovirus (CMV), Epstein-Barr virus (EBV), Kaposi's sarcoma associated-herpesvirus (KSHV), influenzavirus A, H5N1 avian influenza virus, influenzavirus B, influenzavirus C, severe acute respiratory syndrome (SARS) virus, rabies virus, and vesicular stomatitis virus (VSV).

71. The method of claim 69, wherein the bacterium is selected from the group consisting of Pseudomonas aeruginosa, Salmonella typhimurium, Escherichia coli, Klebsiella pneumoniae, Bruscella, Burkholderia mallei, Yersinia pestis, and Bacillus anthracis.

72. The method of claim 69, wherein the fungus is selected from the group consisting of Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, and Rhizopus arrhizus.

73. The method of claim 69, wherein the parasite is selected from the group consisting of Toxoplasma gondii, Plasmodium falciparum, P. vivax, P. ovale, P. malariae, Trypanosoma spp., and Legionella spp.

74. The method of any one of claims 1-59, wherein the disease is selected from the group consisting of hypertension, hyperglycemia, hyperlipidemia, edema, depression, obesity, infertility, amenorrhea, fatigue, vertigo, uterine bleeding, uterine ulcer, hyperthyroidism, myoma, endometriosis, cerebral palsy, brain atrophy, systemic muscular atrophy, trigeminal neuralgia, schizophrenia, epilepsy, amyotrophic lateral sclerosis (ALS), Parkinson's Disease, autism, Alzheimer's Disease, Huntington's Disease, emphysema, asthma, hepatitis B, cough, systemic fibroma, renal diseases, lung diseases, and liver diseases.

75. The method of any one of claims 1-74, wherein the subject is a mammal.

76. The method of claim 75, wherein the mammal is a human.

77. A kit comprising a clamp and a package insert instructing a user of said kit to treat a subject according to the method of any one of claims 1-76.

78. The kit of claim 77, wherein the clamp comprises a regulator screw capable of maintaining a desired distance between arms of said clamp.

79. The kit of claim 78, wherein the regulator screw can be adjusted to one of a plurality of settings.

80. The kit of claim 79, wherein the plurality of settings correspond to a state of maximum distance between the arms of said clamp, one or more states of intermediate distance between the arms of said clamp, and a state of minimum distance between the arms of said clamp.

81. The kit of claim 80, wherein the setting that corresponds to said state of maximum distance between the arms of said clamp is indicated by exposure of a green color adjacent to an arm of said clamp.

82. The kit of claim 80, wherein the setting that corresponds to said state of intermediate distance between the arms of said clamp is indicated by exposure of a yellow color adjacent to an arm of said clamp.

83. The kit of claim 80, wherein the setting that corresponds to said state of minimum distance between the arms of said clamp is indicated by exposure of a red color adjacent to an arm of said clamp.

84. The method of claim 1, wherein the portion of tissue comprises one or more tumors.

85. The method of claim 84, wherein the clamping causes crushing of said one or more tumors.

86. The method of claim 1, wherein the method further comprises compressing one or more muscles and/or connective tissues located underneath said skin.

87. The method of claim 1, wherein the pressure is applied at a location at which said skin converges with surrounding skin that is not folded.

88. The method of claim 1, wherein the clamp comprises curved ends and a flexible handle.

89. The method of claim 88, wherein the flexible handle is configured to allow a user of said clamp to control the duration of pressure applied by said clamping.

90. The method of claim 88 or 89, wherein the flexible handle is configured to allow a user of said clamp to control the amount of pressure applied by said clamping.

91. The method of claim 1, wherein the clamp comprises a mechanism capable of regulating distance between said curved ends.

92. The method of claim 1, wherein the clamp comprises arms and a regulator screw capable of maintaining a desired distance between the arms of the clamp.

93. The method of claim 92, wherein the regulator screw can be adjusted to one of a plurality of settings.

94. The method of claim 93, wherein the plurality of settings correspond to a state of maximum distance between the arms of the clamp, one or more states of intermediate distance between the arms of the clamp, and a state of minimum distance between the arms of the clamp.

95. The method of claim 94, wherein the maximum distance is between about 2.4 cm and about 7.2 cm.

96. The method of claim 94, wherein the intermediate distance is between about 1.6 cm and about 4.8 cm.

97. The method of claim 94, wherein the minimum distance is between about 0.8 and about 2.4 cm.

98. The method of any one of claims 94-97, wherein the setting that corresponds to the state of maximum distance between the arms of the clamp is indicated by exposure of a green color adjacent to an arm of the clamp.

99. The method of claim 94, wherein the setting that corresponds to said state of intermediate distance between the arms of said clamp is indicated by exposure of a yellow color adjacent to an arm of said clamp.

100. The method of claim 94, wherein the setting that corresponds to said state of minimum distance between the arms of said clamp is indicated by exposure of a red color adjacent to an arm of said clamp.

101. The method of claim 1, wherein the clamping occurs for a period of about 1 to about 10 seconds.

102. The method of claim 101, wherein the clamping occurs for a period of about 1 to about 3 seconds.

103. The method of claim 1, wherein the clamping is performed one or more times daily, weekly, monthly, or yearly.

104. The method of claim 103, wherein the clamping is performed one or more times daily.

105. The method of claim 104, wherein the clamping is performed one or more times daily for between about two and about twenty days.

106. The method of claim 105, wherein the clamping is performed one or more times daily for six days.

107. The method of claim 106, wherein at the end of said six days, the clamping is repeated one or more times every other day for a total of 16 additional days to form a standard treatment course.

108. The method of claim 107, wherein the standard treatment course is repeated between two and twenty times.

109. The method of claim 108, wherein the standard treatment course is repeated between five and fifteen times.

110. The method of claim 109, wherein the standard treatment course is repeated between six and nine times.

111. The method of claim 110, wherein the standard treatment course is repeated seven times.

112. The method of claim 110, wherein the standard treatment course is repeated eight times.

113. The method of claim 107, wherein the clamping is performed with increasing pressure over the duration of said standard treatment course.

114. The method of claim 1, wherein the clamping is sufficient to cause pain and/or bruising to said tissue.

115. The method of claim 1, wherein the clamping causes an increase in the amount or concentration of one or more antigens on the surface of a cancer cell within said subject.

116. The method of claim 1, wherein the clamping causes an increase in the amount or concentration of one or more dendritic cells on the surface of a cancer cell within said subject.

117. The method of claim 116, wherein the dendritic cells are located below said skin.

118. The method of claim 1, wherein the subject exhibits an elevated immune response after said clamping is performed.

119. The method of claim 118, wherein the elevated immune response is selected from the group consisting of inflammation, secretion of chemokines, an increase in the level of one or more cytokines, and activation of one or more immune cells within said subject.

120. The method of claim 119, wherein the inflammation comprises one or more processes selected from the group consisting of arteriole dilation, an increase in capillary permeability, and migration of neutrophils and/or macrophages from capillaries or venules into interstitial spaces.

121. The method of claim 119, wherein the chemokines are capable of attracting to said portion of tissue one or more macrophages, T-cells, mast cells, dendritic cells, activated dendritic cells, eosinophils, and/or neutrophils.

122. The method of claim 119, wherein the one or more cytokines are selected from the group consisting of TNFα, IFNγ, IL-1, IL-6, and IL-8.

123. The method of claim 119, wherein the one or more immune cells are selected from the group consisting of macrophages, neutrophils, T-cells, antigen-presenting cells, and dendritic cells.

124. The method of claim 1, wherein the subject exhibits epidermal lumps after said clamping is performed.

125. The method of claim 124, wherein the lumps are between about 1 cm and about 3 cm in length.

126. The method of claim 124 or 125, wherein the method further comprising the step of compressing said lumps.

127. The method of claim 126, wherein the lumps are broken into a plurality of fragments.

128. The method of claim 126 or 127, wherein the lumps comprise cell debris.

129. The method of claim 128, wherein the cell debris is or comprises monosodium urate.

130. The method of claim 129, wherein the monosodium urate is in a crystalline form.

131. The method of claim 129 or 130, wherein the monosodium urate is released within said subject.

132. The method of claim 131, wherein the monosodium urate is released into peripheral tissues within said subject.

133. The method of claim 129, wherein the monosodium urate promotes the growth or maturation of one or more cells selected from the group consisting of macrophages, neutrophils, T-cells, antigen-presenting cells, and dendritic cells.

134. The method of claim 129, wherein the monosodium urate induces the production of IL-1 _{R} in said subject.

135. The method of claim 121, wherein the T-cells are CD4+ or CD8+ T-cells.

136. The method of claim 121, wherein the T-cells are capable of specifically binding an antigen expressed on the surface of a cancer cell.

137. The method of claim 136, wherein the T-cells are capable of killing one or more of said cancer cells.

138. The method of claim 1, wherein the skin is located along a meridian line of said subject.

139. The method of claim 138, wherein the meridian line is selected from the group consisting of wood phase meridian, first fire phase meridian, second fire phase meridian, earth phase meridian, metal phase meridian, water phase meridian, lung meridian, heart meridian, liver meridian, spleen meridian, kidney meridian, pericardium meridian, large intestine meridian, small intestine meridian, stomach meridian, bladder meridian, and gall bladder meridian.

140. The method of claim 1, wherein the disease is a cancer.

141. The method of claim 140, wherein the cancer is selected from the group consisting of leukemia, lymphoma, liver cancer, bone cancer, skin cancer, pulmonary cancer, brain cancer, bladder cancer, gastrointestinal cancer, breast cancer, cardiac cancer, cervical cancer, uterine cancer, head and neck cancer, gallbladder cancer, laryngeal cancer, lip and oral cavity cancer, ocular cancer, melanoma, pancreatic cancer, prostate cancer, colorectal cancer, testicular cancer, throat cancer, adenocarcinoma, pituitary adenoma, acute lymphoblastic leukemia (ALL), acute myeloid leukemia (AML), chronic lymphocytic leukemia (CLL), chronic myelogenous leukemia (CML), adrenocortical carcinoma, AIDS-related lymphoma, primary CNS lymphoma, anal cancer, appendix cancer, astrocytoma, atypical teratoid/rhabdoid tumor, basal cell carcinoma, bile duct cancer, extrahepatic cancer, ewing sarcoma family, osteosarcoma and malignant fibrous histiocytoma, central nervous system embryonal tumors, central nervous system germ cell tumors, craniopharyngioma, ependymoma, bronchial tumors, burkitt lymphoma, carcinoid tumor, primary lymphoma, chordoma, chronic myeloproliferative neoplasms, colon cancer, extrahepatic bile duct cancer, ductal carcinoma in situ (DCIS), endometrioma, ependymoma, esophageal cancer, esthesioneuroblastoma, extracranial germ cell tumor, extragonadal germ cell tumor, fallopian tube cancer, fibrous histiocytoma of bone, gastrointestinal carcinoid tumor, gastrointestinal stromal tumors (GIST), testicular germ cell tumor, gestational trophoblastic disease, glioma, childhood brain stem glioma, hairy cell leukemia, hepatocellular cancer, langerhans cell histiocytosis, hodgkin lymphoma, hypopharyngeal cancer, islet cell tumors, pancreatic neuroendocrine tumors, wilms tumor and other childhood kidney tumors, langerhans cell histiocytosis, small cell lung cancer, cutaneous T-cell lymphoma, intraocular melanoma, merkel cell carcinoma, mesothelioma, metastatic squamous neck cancer, midline tract carcinoma, multiple endocrine neoplasia syndromes, multiple myeloma/plasma cell neoplasm, myelodysplastic syndromes, nasal cavity and paranasal sinus cancer, nasopharyngeal cancer, neuroblastoma, non-hodgkin lymphoma (NHL), non-small cell lung cancer (NSCLC), ovarian carcinoma, low malignant potential ovarian cancer, pancreatic neuroendocrine tumors, papillomatosis, paraganglioma, paranasal sinus and nasal cavity cancer, parathyroid cancer, penile cancer, pharyngeal cancer, pheochromocytoma, pituitary tumor, pleuropulmonary blastoma, primary peritoneal cancer, rectal cancer, retinoblastoma, rhabdomyosarcoma, salivary gland cancer, kaposi sarcoma, rhabdomyosarcoma, sézary syndrome, small intestine cancer, soft tissue sarcoma, throat cancer, thymoma and thymic carcinoma, thyroid cancer, transitional cell cancer of the renal pelvis and ureter, urethral cancer, uterine sarcoma, vaginal cancer, vulvar cancer, and waldenström macroglobulinemia.

142. The method of claim 140, wherein the cancer is selected from the group consisting of breast cancer, adenocarcinoma, leukemia, skin cancer, ovarian carcinoma, pituitary adenoma, pulmonary cancer, endometrioma, and cervical cancer.

143. The method of claim 142, wherein the breast cancer is selected from the group consisting of late stage breast cancer, bilateral breast ductal carcinoma, and invasive bilateral breast cancer.

144. The method of claim 1, wherein the disease is a neoplasm.

145. The method of claim 144, wherein the neoplasm is a growth of a tissue or organ selected the group consisting of a pancreas, salivary gland, pituitary gland, kidney, heart, lung, hematopoietic system, cranial nerves, heart, aorta, olfactory gland, hypopharynx, ear, nerves, structures of the head, eye, thymus, tongue, bone, liver, small intestine, large intestine, gut, brain, skin, peripheral nervous system, central nervous system, spinal cord, breast, embryonic structures, embryos, and testes.

146. The method of claim 145, wherein the neoplasm is a growth of an organ selected from the group consisting of a lung, kidney, and hypopharynx.

147. The method of claim 1, wherein the disease is an autoimmune disease selected from the group consisting of type I diabetes, alopecia areata, ankylosing spondylitis, antiphospholipid syndrome, autoimmune Addison's Disease, autoimmune hemolytic anemia, autoimmune hepatitis, Behcet's Disease, bullous pemphigoid, cardiomyopathy, celiac sprue-dermatitis, chronic fatigue immune dysfunction syndrome (CFIDS), chronic inflammatory demyelinating polyneuropathy, Churg-Strauss Syndrome, cicatricial pemphigoid, crest syndrome, cold agglutinin disease, Crohn's Disease, essential mixed cryoglobulinemia, fibromyalgia-fibromyositis, Graves' Disease, Guillain-Barré Syndrome, Hashimoto's thyroiditis, hypothyroidism, idiopathic pulmonary fibrosis, idiopathic thrombocytopenia purpura (ITP), IgA nephropathy, juvenile arthritis, lichen planus, lupus, systemic lupus erythematosus, Ménière's Disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's Phenomenon, Reiter's Syndrome, rheumatic fever, rheumatoid arthritis, sarcoidosis, scleroderma, Sjögren's Syndrome, stiff-man syndrome, Takayasu Arteritis, temporal arteritis/giant cell arteritis, ulcerative colitis, uveitis, vasculitis, vitiligo, and Wegener's Granulomatosis.

148. The method of claim 147, wherein the autoimmune disease is selected from the group consisting of type I diabetes and lupus.

149. The method of claim 1, wherein the disease is an infectious disease caused by one or more agents selected from the group consisting of a virus, a bacterium, a fungus, or a parasite.

150. The method of claim 149, wherein the virus is selected from the group consisting of Gadgets Gully virus, Kadam virus, Kyasanur Forest disease virus, Langat virus, Omsk hemorrhagic fever virus, Powassan virus, Royal Farm virus, tick-borne encephalitis virus, Louping ill virus, Meaban virus, Saumarez Reef virus, Tyuleniy virus, Aroa virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus, Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, and Rio Bravo virus, Venezuelan equine encephalitis virus (VEE), Eastern equine encephalitis virus (EEE), Western equine encephalitis virus (WEE), Ebola virus, Marburg virus, smallpox virus, vaccinia virus, Lassa virus, Ippy virus, lymphocytic choriomeningitis virus (LCMV), Mobala virus, Mopeia virus, Amapari virus, Flexal virus, Guanarito virus, Junin virus, Latino virus, Machupo virus, Oliveros virus, Paraná virus, Pichinde virus, Pirital virus, Sabiá virus, Tacaribe virus, Tamiami virus, and Whitewater Arroyo virus, Sin Nombre virus, Hantaan virus, Rift Valley fever virus, Crimean-Congo hemorrhagic fever virus, Dugbe virus, herpes simplex virus (HSV), cytomegalovirus (CMV), Epstein-Barr virus (EBV), Kaposi's sarcoma associated-herpesvirus (KSHV), influenzavirus A, H5N1 avian influenza virus, influenzavirus B, influenzavirus C, severe acute respiratory syndrome (SARS) virus, rabies virus, and vesicular stomatitis virus (VSV).

151. The method of claim 149, wherein the bacterium is selected from the group consisting of Pseudomonas aeruginosa, Salmonella typhimurium, Escherichia coli, Klebsiella pneumoniae, Bruscella, Burkholderia mallei, Yersinia pestis, and Bacillus anthracis.

152. The method of claim 149, wherein the fungus is selected from the group consisting of Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, and Rhizopus arrhizus.

153. The method of claim 149, wherein the parasite is selected from the group consisting of Toxoplasma gondii, Plasmodium falciparum, P. vivax, P. ovale, P. malariae, Trypanosoma spp., and Legionella spp.

154. The method of claim 1, wherein the disease is selected from the group consisting of hypertension, hyperglycemia, hyperlipidemia, edema, depression, obesity, infertility, amenorrhea, fatigue, vertigo, uterine bleeding, uterine ulcer, hyperthyroidism, myoma, endometriosis, cerebral palsy, brain atrophy, systemic muscular atrophy, trigeminal neuralgia, schizophrenia, epilepsy, amyotrophic lateral sclerosis (ALS), Parkinson's Disease, autism, Alzheimer's Disease, Huntington's Disease, emphysema, asthma, hepatitis B, cough, systemic fibroma, renal diseases, lung diseases, and liver diseases.

155. The method of claim 1, wherein the subject is a mammal.

156. The method of claim 155, wherein the mammal is a human.

157. A kit comprising a clamp and a package insert instructing a user of said kit to treat a subject according to the method of claim 1.

158. The kit of claim 157, wherein the clamp comprises a regulator screw capable of maintaining a desired distance between arms of said clamp.

159. The kit of claim 158, wherein the regulator screw can be adjusted to one of a plurality of settings.

160. The kit of claim 159, wherein the plurality of settings correspond to a state of maximum distance between the arms of said clamp, one or more states of intermediate distance between the arms of said clamp, and a state of minimum distance between the arms of said clamp.

161. The kit of claim 160, wherein the setting that corresponds to said state of maximum distance between the arms of said clamp is indicated by exposure of a green color adjacent to an arm of said clamp.

162. The kit of claim 160, wherein the setting that corresponds to said state of intermediate distance between the arms of said clamp is indicated by exposure of a yellow color adjacent to an arm of said clamp.

163. The kit of claim 160, wherein the setting that corresponds to said state of minimum distance between the arms of said clamp is indicated by exposure of a red color adjacent to an arm of said clamp.
